SEQUENCE LISTING (13 sequences)

SEQ ID NO: 1 - protein, 11 aa, synthetic construct
GGSISSNYAWN

SEQ ID NO: 2 - protein, 16 aa, synthetic construct
YIYYSGNTNYNPSLKS

SEQ ID NO: 3 - protein, 9 aa, synthetic construct
SYYGNSFIY

SEQ ID NO: 4 - protein, 17 aa, synthetic construct
KSSQSLLNSGNQKNYLT

SEQ ID NO: 5 - protein, 7 aa, synthetic construct
WASTRES

SEQ ID NO: 6 - protein, 9 aa, synthetic construct
QNAYSFPWT

SEQ ID NO: 7 - protein, 118 aa, synthetic construct
QVQLQESGPGLVKPSETLSLTCTVSGGSISSNYAWNWIRQPPGKGLEWIGYIYYSGNTNYNPSLKSRVTISRDTSKNQFSLKLSSVTAADTAVYYCATSYYGNSFIYWGQGTLVTVSS

SEQ ID NO: 8 - protein, 113 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNAYSFPWTFGQGTKVEIK

SEQ ID NO: 9 - protein, 448 aa, synthetic construct
QVQLQESGPGLVKPSETLSLTCTVSGGSISSNYAWNWIRQPPGKGLEWIGYIYYSGNTNYNPSLKSRVTISRDTSKNQFSLKLSSVTAADTAVYYCATSYYGNSFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNAYSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 12 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 13 - protein, 5 aa, synthetic construct
GGGGS

(19) United States
(12) Patent Application Publication
(10) Pub. No.: US 2026/0199509 A1
(43) Pub. Date: Jul. 16, 2026

(54) BILIARY TRACT CANCER TREATMENT WITH ANTI-CLAUDIN 18.2 ADC

(71) Applicant: ASTRAZENECA AB, Södertälje (SE)

(72) Inventors: Alejandra NEGRO, Wilmington, DE (US); Osama Entoun RAHMA, Wilmington, DE (US); James M. DUNLEAVEY, Wilmington, DE (US); Mel EHUDIN, Wilmington, DE (US); Marco GYMNOPOULOS, Wilmington, DE (US)

(21) Appl. No.: 19/413,978

(22) Filed: Dec. 9, 2025

Related U.S. Application Data
(60) Provisional application No. 63/729,822, filed on Dec. 9, 2024.

(51) Int. Cl. A61K 47/68; A61P 35/00; C07K 16/28; G01N 33/575

(52) U.S. Cl. CPC A61K 47/6863; A61K 47/68031; A61K 47/6889; A61P 35/00; C07K 16/28; G01N 33/5759; C07K 2317/565; G01N 2800/50; G01N 2800/52

(57) ABSTRACT

Provided herein is a method of treating biliary tract cancers in humans. The method uses an antibody-drug conjugate (ADC) comprising an antibody (or antigen-binding fragment thereof) which specifically binds claudin 18.2 (CLDN18.2). The ADC is used for treatment of biliary tract cancers which express CLDN18.2.

### SEQUENCE LISTING

The content of the electronically submitted sequence listing (Name: 8887515.xml; Size: 13,435 bytes; Date of Creation: Dec. 5, 2025) filed with the application is incorporated herein by reference in its entirety.

### FIELD

Provided herein are methods by which biliary tract cancer (BTC) patients susceptible to treatment with an antibody-drug conjugate (ADC) comprising an anti-claudin 18.2 (anti-CLDN18.2) antibody can be identified and treated.

### BACKGROUND

The Claudin (CLDN) proteins participate in the formation of tight junctions between epithelial cells. CLDN proteins are transmembrane proteins with 4 transmembrane helices, with cytoplasmic N- and C-termini and two extracellular loops (ECLs). Different members of the Claudin family are expressed in different tissues.

Claudin 18 has two isoforms produced as a result of alternative splicing: CLDN18.1 and CLDN18.2. In healthy individuals, CLDN18.1 is expressed primarily in the lungs, in alveolar epithelial cells, whereas CLDN18.2 is expressed primarily in the stomach, in gastric mucosal membrane epithelial cells.

However, CLDN18.2 has been found to be expressed in a variety of cancers, including about 70% of gastric cancers, 50% of pancreatic cancers and approximately 65% of biliary tract cancers (BTCs) (Xiao Yi et al., 2023). Moreover, in healthy individuals CLDN18.2 is buried in tight junctions, but during malignant transformation is believed to become more exposed and thus therapeutically accessible. As a result, CLDN18.2 has been identified as a target for treatment of these cancers, including biliary tract cancer.

Biliary tract cancer (BTC) is a group of adenocarcinomas that arise from the epithelial cell lining of the bile ducts (cholangiocarcinoma) and the gallbladder. For localised BTC, surgical resection can be curative.

However, it is often challenging to diagnose BTC at an early stage due to its anatomical location and the lack of disease-specific symptoms, and thus >75% of patients with BTC present with advanced, unresectable disease at first diagnosis (Lamarca et al., 2014). In addition, BTC patients often relapse even after curative surgery and the prognosis for BTC is dismal, with estimated 5-year overall survival (OS) rates of 10% to 33% for localised disease and 1% to 2% for metastatic disease (Caparica et al., 2019; Lamarca et al., 2014; Siegal et al., 2020).

Systemic therapy is a primary treatment option for unresectable or metastatic disease, with studies reporting an improved survival benefit with durvalumab (a PD-L1 inhibitor) in combination with cisplatin and gemcitabine. More recently, the role of immunotherapy in improving OS in the first line advanced BTC setting was further confirmed using gemcitabine plus cisplatin in combination with pembrolizumab (Kelly et al., 2023). However, most patients receiving front-line chemotherapy with or without PD-1/PD-L1 inhibitor eventually become refractory to therapy or subsequently experience disease progression. Fluoropyrimidine and irinotecan- or oxaliplatin-based combination chemotherapy regimens have been widely used in clinical practice as the second line treatment for locally advanced or metastatic BTC. However, their efficacy is modest with an overall response rate (ORR) of 8% to 15% (Lamarca et al., 2021; Mizrahi et al., 2020; Yoo et al., 2021).

In addition, patients with clinically relevant molecular alterations may benefit from biomarker-lead targeted therapies such as FGFR and IDH-1 inhibitors. However, these targeted BTC therapies only cater to a small, biomarker-positive patient population in BTC, mostly in locally advanced/metastatic intra-hepatic cholangiocarcinoma, and thus only a limited number of patients are able to receive these treatment options. Consequently, there is a high unmet medical need to develop effective and tolerable therapies for patients with locally advanced/metastatic BTC who have progressed after frontline systemic therapy.

CLDN18.2-targeting ADCs have been tested in small cohorts of BTC patients (Yu et al., 2024; Bai et al., 2024), including in combination with the anti-PD-1/CTLA-4 bispecific antibody cadonilimab (Fan et al., 2024).

WO 2020/211792 discloses a number of anti-CLDN18.2 antibodies that can be used in cancer therapy, including CM311. Antibody-drug conjugates (ADCs) based on the same antibody are disclosed in WO 2022/078523, including an ADC comprising CM311 conjugated to the potent antineoplastic agent monomethyl auristatin E (MMAE), known as CMG901 or AZD0901. A phase 1 clinical trial of CMG901 in gastric, gastroesophageal junction and pancreatic cancer patients has shown promising preliminary results (Xu et al., 2023), and a phase 3 clinical trial of CMG901 for treatment of gastric cancer and gastroesophageal junction cancer is ongoing (NCT06346392).

### SUMMARY

The present disclosure relates to the treatment of BTC with an anti-CLDN18.2 ADC, in particular the ADC CMG901.

In a first aspect, provided herein is a method of treating biliary tract cancer (BTC) in a human subject, comprising administering to the subject a therapeutically effective dose of an antibody-drug conjugate (ADC) comprising an antibody or antigen-binding fragment thereof that specifically binds claudin 18.2 (CLDN18.2) conjugated to a cytotoxic agent,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
   and wherein the biliary tract cancer expresses CLDN18.2.

Relatedly, provided herein is an antibody-drug conjugate (ADC) comprising an antibody or antigen-binding fragment thereof that specifically binds claudin 18.2 (CLDN18.2) conjugated to a cytotoxic agent for use in a method of treating biliary tract cancer (BTC) in a human subject,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
   and wherein the biliary tract cancer expresses CLDN18.2.

Also relatedly, provided herein is the use of an antibody-drug conjugate (ADC) comprising an antibody or antigen-binding fragment thereof that specifically binds claudin 18.2 (CLDN18.2) conjugated to a cytotoxic agent to treat biliary tract cancer (BTC) in a human subject,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
   and wherein the biliary tract cancer expresses CLDN18.2.

Also relatedly, provided herein is the use of an antibody-drug conjugate (ADC) comprising an antibody or antigen-binding fragment thereof that specifically binds claudin 18.2 (CLDN18.2) conjugated to a cytotoxic agent in the manufacture of a medicament for treating biliary tract cancer (BTC) in a human subject,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
   and wherein the biliary tract cancer expresses CLDN18.2.

Also relatedly, provided herein is a pharmaceutical composition for use in treating biliary tract cancer (BTC) in a human subject, wherein the composition comprises an antibody-drug conjugate (ADC) comprising an antibody or antigen-binding fragment thereof that specifically binds claudin 18.2 (CLDN18.2) conjugated to a cytotoxic agent,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
   and wherein the biliary tract cancer expresses CLDN18.2.

In a second aspect, provided herein is a method of identifying and treating a human subject with biliary tract cancer susceptible to treatment with an ADC as defined in the first aspect, wherein the method comprises:
(a) determining whether the biliary tract cancer expresses CLDN18.2;
(b) identifying the subject as susceptible to treatment with the ADC if the biliary tract cancer expresses CLDN18.2; and
(c) when the subject is identified as susceptible to treatment with the ADC, administering an effective amount of the ADC to the subject.

In a third aspect, provided herein is a method of selecting a human subject with biliary tract cancer for treatment with an ADC as defined in the first aspect, the method comprising assessing the level of CLDN18.2 expression in a tissue sample from the biliary tract cancer, wherein the subject is selected for treatment with the ADC when the biliary tract cancer expresses CLDN18.2.

In a fourth aspect, provided herein is a method of predicting whether a human subject with biliary tract cancer is likely to respond to treatment with an ADC as defined in the first aspect, the method comprising assessing the level of CLDN18.2 expression in a tissue sample from the biliary tract cancer, wherein the subject is considered likely to respond to treatment with the ADC when the biliary tract cancer expresses CLDN18.2.

In a fifth aspect, provided herein is a method for identifying a human subject with biliary tract cancer who is likely to respond to treatment with an ADC as defined in the first aspect, the method comprising obtaining a tissue sample of the biliary tract cancer from the subject and assessing the level of CLDN18.2 expression in the sample, wherein the subject is considered likely to respond to treatment with the ADC when the biliary tract cancer expresses CLDN18.2.

In a sixth aspect, provided herein is a method for assessing the susceptibility of a biliary tract cancer in a human subject to treatment with an ADC as defined in the first aspect, comprising assessing the level of CLDN18.2 expression in a tissue sample from the biliary tract cancer, wherein the biliary tract cancer is considered susceptible to treatment with the ADC when the biliary tract cancer expresses CLDN18.2.

### DETAILED DESCRIPTION

Provided herein are methods for treating CLDN18.2-positive biliary tract cancer with an ADC targeted against CLDN18.2.

### Antibodies

The ADC for use herein comprises an antibody, or antigen-binding fragment thereof, that specifically binds to CLDN18.2.

As defined herein, in line with standard terminology in the art, an antibody is an antigen-binding protein comprising two heavy chains and two light chains. The light chains are shorter (and thus lighter) than the heavy chains. The heavy chains comprise an N-terminal heavy chain variable domain (V_{H}), and the light chains comprise an N-terminal light chain variable domain (V_{L}). The heavy and light chains each comprise constant domains C-terminal to the respective variable domain. Each light chain of an antibody comprises one constant domain (C_{L}), whilst the heavy chains of antibodies of the IgA, IgG, and IgD isotypes contain three constant domains (C_{H}1-C_{H}3), and the heavy chains of antibodies of the IgM and IgE isotypes contain four constant domains (C_{H}1-C_{H}4).

Both the light and heavy chains of an antibody comprise three hypervariable complementarity-determining regions (CDRs), as set out here below. In a pair of a light chain and a heavy chain, the CDRs of the two chains form the antigen-binding site. The CDR sequences determine the specificity of an antibody. The three CDRs of a heavy chain are known as V_{H}CDR1, V_{H}CDR2 and V_{H}CDR3, from N-terminus to C-terminus, and the three CDRs of a light chain are known as V_{L}CDR1, V_{L}CDR2 and V_{L}CDR3, from N-terminus to C-terminus. Framework regions are located in between the CDRs and between the CDRs and ends of the variable domains. Antigen-binding fragments of antibodies are fragments or synthetic constructs comprising one or more antigen-binding sites of an antibody, but not the entire antibody. Generally, an antigen-binding fragment of an antibody comprises the entire V_{L} and V_{H} domain sequences, but lacks at least part of the heavy and/or light chain constant domains.

The antibody, or antigen-binding fragment thereof (henceforth simply “fragment thereof”, or “antibody fragment”), used in the ADC specifically binds human CLDN18.2. Human CLDN18.2 has the UniProt accession number P56856-2, and the amino acid sequence set forth in SEQ ID NO: 11. Thus the antibody or fragment thereof specifically binds a protein with the sequence of SEQ ID NO: 11.

An antibody which binds specifically to human CLDN18.2 is an antibody which binds to human CLDN18.2 with a greater affinity than that with which it binds to other molecules, or at least most other molecules. In particular, an antibody which binds specifically to human CLDN18.2 either does not bind human CLDN18.1 (UniProt P56856-1, SEQ ID NO: 12), or binds human CLDN18.1 with much lower affinity than human CLDN18.2, e.g. it may bind human CLDN18.2 with an affinity which is one or more orders of magnitude higher than the affinity with which it binds human CLDN18.1.

An antibody which specifically binds human CLDN18.2 may display cross-reactivity with CLDN18.2 from other species. Regardless, the skilled person can readily determine whether an antibody or fragment thereof specifically binds human CLDN18.2 using standard techniques in the art, e.g. ELISA, Western-blot, surface plasmon resonance (SPR), etc.

The antibody or fragment thereof used in the present ADC comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises or consists of the amino acid sequence set forth in SEQ ID NO: 1 (GGSISSNYAWN);
V_{H}CDR2 comprises or consists of the amino acid sequence set forth in SEQ ID NO: 2 (YIYYSGNTNYNPSLKS);
V_{H}CDR3 comprises or consists of the amino acid sequence set forth in SEQ ID NO: 3 (SYYGNSFIY);
V_{L}CDR1 comprises or consists of the amino acid sequence set forth in SEQ ID NO: 4 (KSSQSLLNSGNQKNYLT);
V_{L}CDR2 comprises or consists of the amino acid sequence set forth in SEQ ID NO: 5 (WASTRES); and
V_{L}CDR3 comprises or consists of the amino acid sequence set forth in SEQ ID NO: 6 (QNAYSFPWT).

The antibody or fragment thereof used in the ADC for use herein is capable of mediating endocytosis of CLDN18.2 expressed on the surface of a target cell. That is to say, upon binding of the antibody or fragment thereof to CLDN18.2 on the surface of a cell, the CLDN18.2 and bound antibody are internalised into the cell by endocytosis. Whether an antibody induces endocytosis upon binding to a cell surface protein can be determined by e.g. confocal microscopy using a fluorescent-tagged antibody.

As set out above, the present ADC comprises an antibody or fragment thereof. An “antibody” as referred to herein is an immunoglobulin having the features described hereinbefore. Antigen-binding fragments of antibodies are discussed in Rodrigo et al., 2010. A Fab fragment consists of the antigen-binding domain of an antibody, i.e. an individual antibody may be seen to contain two Fab fragments, each consisting of a light chain and its conjoined N-terminal section of the heavy chain. Thus, a Fab fragment contains an entire light chain and the V_{H} and C_{H}1 domains of the heavy chain to which it is bound. Fab fragments may be obtained by digesting an antibody with papain.

F(ab′)2 fragments consist of the two Fab fragments of an antibody, plus the hinge regions of the heavy domains, including the disulphide bonds linking the two heavy chains together. In other words, a F(ab′)2 fragment can be seen as two covalently joined Fab fragments. F(ab′)2 fragments may be obtained by digesting an antibody with pepsin.

Reduction of F(ab′)2 fragments yields two Fab′ fragments, which can be seen as Fab fragments containing an additional sulfhydryl group which can be useful for conjugation of the fragment to other molecules.

Fv fragments consist of just the variable domains of the light and heavy chains. These are not covalently linked and are held together only weakly by non-covalent interactions. Fv fragments can be modified to produce a synthetic construct known as a single chain Fv (scFv) molecule. Such a modification is typically performed recombinantly, by engineering the antibody gene to produce a fusion protein in which a single polypeptide comprises both the V_{H} and V_{L} domains. scFv fragments generally include a peptide linker covalently joining the V_{H} and V_{L} regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is Gly₄Ser (Gly-Gly-Gly-Gly-Ser, SEQ ID NO: 13). Multimers of such linkers may be used, such as for example a dimer, a trimer, a tetramer or a pentamer, e.g. (Gly₄Ser)₂, (Gly₄Ser)₃, (Gly₄Ser)₄ or (Gly₄Ser)₅. However, it is not essential that a linker be present, and the V_{L} domain may be linked to the V_{H} domain by a peptide bond. An scFv is herein defined as an antibody fragment, or antigen-binding fragment of an antibody.

The antibody or antibody fragment used in the ADC may be humanised. A “humanised” antibody is an antibody derived from non-human germline immunoglobulin sequences, but which has been modified to replace non-human sequences with human ones. A humanised antibody may be derived, for instance, from mouse, rat, rabbit, etc., germline immunoglobulin sequences. Indeed, a humanised antibody may be derived from the germline immunoglobulin sequences of any non-human animal. As defined herein, an antibody is considered humanised if at least one of the V_{H} and V_{L} domains is humanised. In particular, a humanised antibody may comprise a humanised V_{H} sequence and a humanised V_{L} sequence.

In a humanised variable domain, a non-human variable domain sequence is modified to replace the non-human (e.g. murine) framework sequences with human framework sequences, such that, generally, the only non-human sequences in the antibody are the CDR sequences (though the CDR sequences may also be modified during the humanisation process). Antibody humanisation is generally performed by a process known as CDR grafting, though any other technique in the art may be used. CDR grafting is well described in Williams, et al., 2010. In this process, humanisation of non-human variable domains involves intercalating the non-human CDRs from each immunoglobulin chain within the framework regions of the most appropriate human variable region. This is done by aligning the non-human variable domains with databases of known human variable domains (e.g. IMGT or Kabat). Appropriate human framework regions are identified from the best aligned variable domains, e.g. domains with high sequence identity between the human and non-human framework regions, domains containing CDRs of the same length, domains having the most similar structures (based on homology modelling), etc. The non-human CDR sequences are then grafted into the lead human framework sequences at the appropriate locations using recombinant DNA technology, and the humanised antibodies then produced and tested for binding to the target antigen. The process of antibody humanisation is known and understood by the skilled individual, who can perform the technique without further instruction. Antibody humanisation services are also offered by a number of commercial companies, e.g. GenScript (USA/China) and LifeArc (UK). Humanised antibody fragments can be easily obtained from humanised antibodies, as described above.

In the present case, the CDR sequences of SEQ ID NOS: 1-6 were originally derived from a murine antibody (though are slightly modified compared to the murine parent antibody). An antibody comprising the CDR sequences of SEQ ID NOS: 1-6 grafted into human framework sequences is a humanised antibody. Thus, the antibody used in the ADC may comprise a V_{L} and/or a V_{H} comprising human framework sequences.

In particular embodiments, the antibody or fragment thereof comprises:
(a) a heavy chain variable domain comprising or consisting of the amino acid sequence set forth in SEQ ID NO: 7, or a variant thereof having at least 80%, 85%, 90% or 95% identity to SEQ ID NO: 7; and
(b) a light chain variable domain comprising or consisting of the amino acid sequence set forth in SEQ ID NO: 8, or a variant thereof having at least 80%, 85%, 90% or 95% identity to SEQ ID NO: 8.

In particular, the antibody or fragment thereof may comprise a heavy chain variable domain comprising or consisting of the amino acid sequence set forth in SEQ ID NO: 7, and a light chain variable domain comprising or consisting of the amino acid sequence set forth in SEQ ID NO: 8.

When the antibody or antigen-binding fragment thereof comprises a V_{H} with at least 80% identity to SEQ ID NO: 7, but less than 100% identity to SEQ ID NO: 7, this is subject to the proviso that the CDRs are as defined above, i.e. that V_{H}CDR1 comprises or consists of the amino acid sequence of SEQ ID NO: 1, V_{H}CDR2 comprises or consists of the amino acid sequence of SEQ ID NO: 2 and V_{H}CDR3 comprises or consists of the amino acid sequence of SEQ ID NO: 3. That is to say, when the heavy chain variable region comprises a variant of SEQ ID NO: 7, all variation in the heavy chain variable domain sequence relative to SEQ ID NO: 7 is found within the framework regions.

Similarly, when the antibody or antigen-binding fragment thereof comprises a V_{L} with at least 80% identity to SEQ ID NO: 8, but less than 100% identity to SEQ ID NO: 8, this is subject to the proviso that the CDRs are as defined above, i.e. that V_{L}CDR1 comprises or consists of the amino acid sequence of SEQ ID NO: 4, V_{L}CDR2 comprises or consists of the amino acid sequence of SEQ ID NO: 5 and V_{L}CDR3 comprises or consists of the amino acid sequence of SEQ ID NO: 6. That is to say, when the light chain variable domain comprises a variant of SEQ ID NO: 8, all variation in the light chain variable domain sequence relative to SEQ ID NO: 8 is found within the framework regions.

In embodiments in which the ADC comprises an antibody, the heavy and light chains of the antibody each comprise a constant region. The constant regions are generally human constant regions.

As is well known in the art, antibodies may belong to a number of different isotypes, with the isotype of an antibody being determined by the sequence of its heavy chain constant region. In humans, the antibody isotypes are IgG, IgE, IgM, IgA and IgD. Some isotypes may be divided into further subtypes, e.g. there are four sub-types of IgG antibodies: IgG1, IgG2, IgG3 and IgG4. When an antibody is used in the methods herein, the antibody may be of any isotype, i.e. an IgG, IgE, IgM, IgA or IgD antibody may be used. In particular, the antibody may be an IgG antibody. When an IgG antibody is used it may be of any sub-type, i.e. IgG1, IgG2, IgG3 or IgG4 antibody may be used. In particular, the antibody may be an IgG1 antibody. In certain embodiments, the antibody is of the human IgG1 isotype (i.e. the antibody may comprise a human IgG1 constant domain).

In humans, the light chain constant region of an antibody may be a kappa or lambda (K or A) constant region, and thus a human antibody light chain may be a K or A light chain. An antibody used herein may comprise a k or A light chain. In particular, the antibody may comprise a k light chain. In certain embodiments, the antibody comprises a light chain comprising a human K constant domain.

The antibody for use herein may comprise:
(i) a heavy chain comprising or consisting of the amino acid sequence set forth in SEQ ID NO: 9, or a variant thereof having at least 80%, 85%, 90% or 95% sequence identity thereto; and
(ii) a light chain comprising or consisting of the amino acid sequence set forth in SEQ ID NO: 10, or a variant thereof having at least 80%, 85%, 90% or 95% sequence identity thereto.

In particular, the antibody or fragment thereof may comprise a heavy chain comprising or consisting of the amino acid sequence set forth in SEQ ID NO: 9, and a light chain comprising or consisting of the amino acid sequence set forth in SEQ ID NO: 10.

As set out above, when the antibody comprises a heavy chain which is a variant of SEQ ID NO: 9, this is subject to the proviso that the CDRs are as defined above, i.e. that V_{H}CDR1 comprises the amino acid sequence of SEQ ID NO: 1, V_{H}CDR2 comprises the amino acid sequence of SEQ ID NO: 2 and V_{H}CDR3 comprises the amino acid sequence of SEQ ID NO: 3. That is to say, when the heavy chain comprises a variant of SEQ ID NO: 9, all variation in the heavy chain sequence relative to SEQ ID NO: 9 is found within the constant domain and the framework regions of the variable domain.

Similarly, when the antibody comprises a light chain which is a variant of SEQ ID NO: 10, this is subject to the proviso that the CDRs are as defined above, i.e. that V_{L}CDR1 comprises the amino acid sequence of SEQ ID NO: 4, V_{L}CDR2 comprises the amino acid sequence of SEQ ID NO: 5 and V_{L}CDR3 comprises the amino acid sequence of SEQ ID NO: 6. That is to say, when the light chain comprises a variant of SEQ ID NO: 10, all variation in the light chain sequence relative to SEQ ID NO: 10 is found within the constant domain and the framework regions of the variable domain.

The antibody with the heavy chain amino acid sequence set forth in SEQ ID NO: 9 and the light chain amino acid sequence SEQ ID NO: 10 is referred to herein as CM311.

Sequence identity of variants of the sequences set out above may be assessed by any convenient method. However, for determining the degree of sequence identity between sequences, computer programmes that make pairwise or multiple alignments of sequences are useful, for instance EMBOSS Needle or EMBOSS stretcher (Rice, et al., 2000) may be used for pairwise sequence alignments while Clustal Omega (Sievers et al., 2011) or MUSCLE (Edgar, 2004) may be used for multiple sequence alignments, though any other appropriate programme may be used. Whether the alignment is pairwise or multiple, it must be performed globally (i.e. across the entirety of the reference sequence) rather than locally.

Sequence alignments and percentage (%) identity calculations may be determined using for instance standard Clustal Omega parameters: matrix Gonnet, gap opening penalty 6, gap extension penalty 1.

Alternatively, the standard EMBOSS Needle parameters may be used: matrix BLOSUM62, gap opening penalty 10, gap extension penalty 0.5. Any other suitable parameters may alternatively be used.

For the purposes of this application, where there is dispute between sequence identity values obtained by different methods, the value obtained by global pairwise alignment using EMBOSS Needle with default parameters shall be considered valid.

Variants of the sequences set out herein (i.e. sequences with at least 80% sequence identity to SEQ ID NO: 7, 8, 9 or 10) may be obtained by substitution, deletion or insertion of amino acid residues relative to the original sequences.

When a sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid in the same family. However, a substitution of an amino acid residue may equally be a non-conservative substitution, in which one amino acid is substituted for another with a sidechain belonging to a different family.

Where an antibody or fragment thereof is used which comprises a variant of the CM311 variable domain sequences (i.e. a variable domain which is a variant of SEQ ID NO: 7 or SEQ ID NO: 8, as set out above) or full chain sequences (i.e. a heavy or light chain which is a variant of SEQ ID NO: 9 or SEQ ID NO: 10), the variant may have equivalent activity to CM311 or a corresponding fragment thereof. For instance, an antibody which is a variant of CM311 (i.e. an antibody comprising a variant sequence as described above) may bind CLDN18.2 with an affinity which is equivalent to CM311, which is not lower than the affinity with which CM311 binds CLDN18.2, or which is not substantially lower than the affinity with which CM311 binds CLDN18.2. For instance, a variant of CM311 may be considered to bind CLDN18.2 with an affinity which is not substantially lower than the affinity with which CM311 binds CLDN18.2 if the variant of CM311 binds CLDN18.2 with an affinity which is reduced by no more than 5%, 10%, 15%, 20% or 25% compared to that of CM311.

The antibody or fragment thereof for use herein may be synthesised by any method known in the art. In particular, the antibody or fragment thereof may be synthesised using a protein expression system, such as a cellular expression system using prokaryotic (e.g. bacterial) host cells or eukaryotic (e.g. yeast, fungus, insect or mammalian) host cells. Cells which may be used in the production of the antibody or fragment thereof are discussed further below. An alternative protein expression system is a cell-free, in vitro expression system, in which a nucleotide sequence encoding the specific binding molecule is transcribed into mRNA, and the mRNA translated into a protein, in vitro. Cell-free expression system kits are widely available, and can be purchased from e.g. ThermoFisher Scientific (USA). Alternatively, antibodies and fragments thereof may be chemically synthesised in a non-biological system. Liquid-phase synthesis or solid-phase synthesis may be used to generate polypeptides which may form or be comprised within the antibody or fragment thereof used herein. The skilled person can readily produce antibodies or fragments thereof using appropriate methodology common in the art.

In particular, the antibody or fragment thereof may be recombinantly expressed in mammalian cells, such as CHO cells. Other suitable mammalian cells for production of the antibody or fragment thereof for use herein include monkey kidney cells (e.g. COS-7), HEK293 Hela cells, baby hamster kidney (BHK) cells, human hepatocellular carcinoma cells (e.g. Hep G2), and a number of other cell lines including the mouse myeloma cell lines NSO and SP2/0.

The host cell, when cultured under appropriate conditions, synthesises the antibody or antigen-binding fragment thereof for use herein that can subsequently be collected from the culture medium (if the host cell secretes it into the medium) or directly from the host cell producing it (if it is not secreted). Thus, the antibody or fragment thereof may be isolated from synthesis, as discussed above. The host cell line which produces the antibody or fragment thereof for use herein may stably express the antibody or fragment thereof or transiently express the antibody or fragment thereof.

### Antibody-Drug Conjugates

The ADC for use herein comprises an antibody or fragment thereof, as described above, conjugated to a cytotoxic agent. That is to say, the antibody or fragment thereof is covalently joined to a cytotoxic agent. The cytotoxic agent may be referred to as the “payload” of the ADC. Throughout this section describing the ADC, the term “antibody” encompasses antibody fragments.

In line with its standard definition in the art, a cytotoxic agent, or cytotoxin, is an agent (or compound) which is toxic to cells. In the context of the ADC used herein, the cytotoxic agent is generally toxic to human cells. By “toxic to cells” is meant that the agent, when delivered to a cell, induces cell death, e.g. by apoptosis or necrosis.

An array of cytotoxic agents suitable for use as ADC warheads is known in the art. In particular embodiments, the cytotoxic agent is an antineoplastic drug. Examples of suitable antineoplastic drugs for use herein include SN-38, gemcitabine, monomethyl auristatin E (MMAE), monomethyl auristatin F (MMAF), doxorubicin, calicheamicin, duocarmycins and maytansinoids (e.g. maytansine DM1, also referred to as mertansine; and maytansine DM4, also referred to as ravtansine), which are well known in the art.

For example, SN-38 has the structure set forth in Formula I:

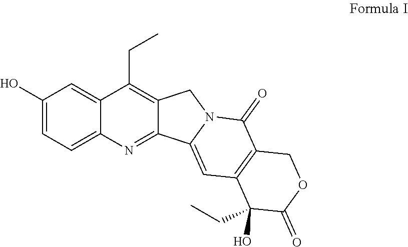 [SMILES: CCc1c2c(nc3ccc(O)cc13)-c1cc3c(c(=O)n1C2)COC(=O)[C@]3(O)CC]

Gemcitabine has the structure set forth in Formula II:

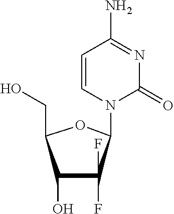

MMAE has the structure set forth in Formula III (in which the wavey line indicates the antibody or fragment thereof to which MMAE is bound, optionally including a linker between the antibody or antibody fragment and the MMAE):

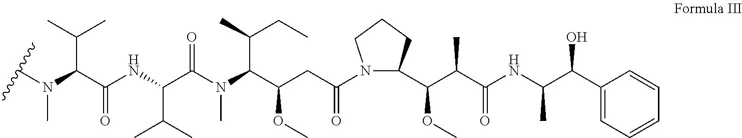 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

MMAF has the structure set forth in Formula IV:

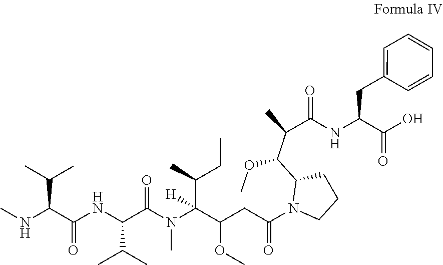 [SMILES: CC[C@H](C)[C@@H](C(CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

Doxorubicin has the structure set forth in Formula V:

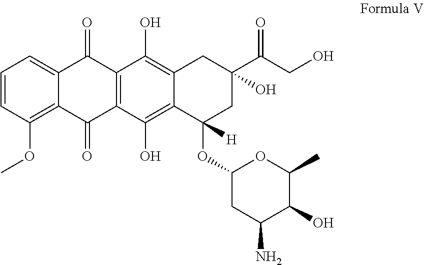 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

In other embodiments, the cytotoxic agent may be a toxin, e.g. a bacterial toxin, such as diphtheria toxin (produced by Corynebacterium diphtheriae) which has the UniProt accession number P00588; or a plant toxin, such as ricin (produced by the castor oil plant Ricinus communis), which has the UniProt accession number P02879.

In particular embodiments, the cytotoxic agent is MMAE. MMAE is a tubulin polymerisation inhibitor which is used in a number of existing approved ADCs, e.g. brentuximab vedotin and tisotumab vedotin. The term “vedotin” is used to refer to MMAE in the context of an ADC. Use of an ADC comprising the CM311 antibody conjugated to MMAE is described in the examples below.

The cytotoxic agent, e.g. MMAE, may be joined to the antibody by a linker. A linker, as defined herein, is any chemical group or entity (which may be a peptide) which joins the cytotoxic agent to the antibody.

The linker may join the cytotoxic agent to any functional group on the antibody, e.g. an amino group, carboxyl group, hydroxyl group or thiol group. The linker may join the cytotoxic agent to a side chain of the antibody or to a terminus of an antibody chain. Generally, the cytotoxic agent is joined to the antibody via thiol groups.

Linkers suitable for use in ADCs are known in the art, and any suitable linker may be used herein. In some embodiments, the linker is protease-cleavable. That is to say, the linker may be susceptible to cleavage by an intracellular protease, particularly an endosomal or lysosomal protease, such that upon endocytosis of the ADC by a target cell, the linker is cleaved and the cytotoxic agent (e.g. MMAE) released. In some embodiments, the linker is a cathepsin-cleavable linker, i.e. a linker susceptible to cleavage by a cathepsin protease, e.g. cathepsin B. Where the cytotoxic agent is MMAE, following release from the antibody it is transported into the cytoplasm where it binds to tubulin and inhibits its polymerization, thereby blocking mitosis, inhibiting tumour cell proliferation and leading to tumour cell death.

In some embodiments, the linker is selected from 6-maleimidohexanoyl (MC), maleimidopropionyl (MP), N-succinimidyl 4-(2-pyridylthio) valerate (SPP), 4-(N-maleimidomethyl)-cyclohexan-1-formyl (MCC), N-succinimidyl(4-iodo-acetyl)aminobenzoate (SIAB), and 6-maleimidocaproyl-valine-citrulline-p-aminobenzyloxycarbonyl (MC-vc-PAB). In particular embodiments, the linker is MC-vc-PAB. MC-vc-PAB has the structure set out in Formula VI below:

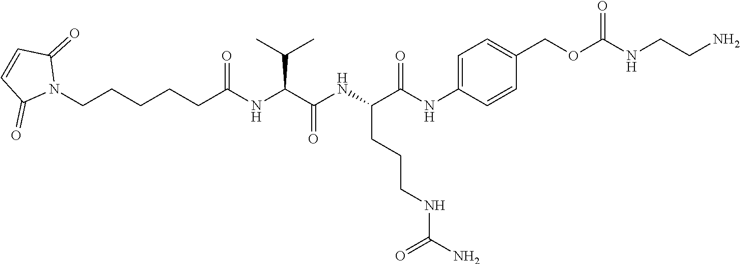 [SMILES: CC(C)[C@H](NC(=O)CCCCCN1C(=O)C=CC1=O)C(=O)N[C@@H](CCCNC(N)=O)C(=O)Nc1ccc(COC(=O)NCCN)cc1]

In some embodiments, the ADC comprises an antibody or fragment thereof as described above conjugated to MMAE by a 6-maleimidocaproyl-valine-citrulline-p-aminobenzyloxycarbonyl (MC-vc-PAB) linker.

In some embodiments, the ADC comprises an antibody or fragment thereof comprising the heavy chain variable domain of SEQ ID NO: 7 and the light chain variable domain of SEQ ID NO: 8, conjugated to a cytotoxic agent by a MC-vc-PAB linker.

In some embodiments, the ADC comprises an antibody or fragment thereof comprising the heavy chain variable domain of SEQ ID NO: 7 and the light chain variable domain of SEQ ID NO: 8, conjugated to MMAE by a MC-vc-PAB linker.

In some embodiments, the ADC comprises an antibody comprising the heavy chain of SEQ ID NO: 9 and the light chain of SEQ ID NO: 10, conjugated to a cytotoxic agent by a MC-vc-PAB linker.

In some embodiments, the ADC comprises an antibody comprising the heavy chain of SEQ ID NO: 9 and the light chain of SEQ ID NO: 10, conjugated to MMAE by a MC-vc-PAB linker. This ADC is referred to herein as AZD0901 or CMG901.

The antibody in the ADC may be conjugated to e.g. 3, 4 or 5 molecules of the cytotoxic agent (e.g. MMAE molecules). The number of molecules of the cytotoxic agent attached to the antibody in the ADC is referred to as the drug-antibody ratio (DAR). It should be noted that the ADC used herein is generally provided in a pharmaceutical composition containing multiple ADC molecules, which may have the same or different DAR values. That is to say, in some embodiments, provided herein are pharmaceutical compositions comprising the ADC described above, in which the ADC molecules all comprise the same number of cytotoxic agent moieties. In other embodiments, provided herein are pharmaceutical compositions comprising the ADC described above, in which the ADC molecules comprise different numbers of cytotoxic agent moieties.

The average number of cytotoxic agent moieties in the ADC molecules in a composition thereof is referred to herein as the average DAR. This may be referred to as the average DAR of a composition comprising the ADC, or as the average DAR of the ADC (i.e. reference to the average DAR of the ADC is shorthand for reference to the average DAR of a composition comprising the ADC, i.e. a composition comprising a population of the ADC). The average DAR for a composition of the ADC can be determined by any conventional means in the art, e.g. mass spectrometry or HPLC. WO 2022/078523 describes determination of the average DAR of a composition of the ADC by hydrophobic interaction chromatography-HPLC (HIC-HPLC).

In some embodiments, the ADC has an average DAR of 3 to 4.5, e.g. 3 to 4.4, 3 to 4.3, 3 to 4.2, 3 to 4.1, 3 to 4, 3.1 to 4.5, 3.1 to 4.4, 3.1 to 4.3, 3.1 to 4.2, 3.1 to 4.1, 3.1 to 4, 3.2 to 4.5, 3.2 to 4.4, 3.2 to 4.3, 3.2 to 4.2, 3.2 to 4.1, 3.2 to 4, 3.3 to 4.5, 3.3 to 4.4, 3.3 to 4.3, 3.3 to 4.2, 3.3 to 4.1, 3.3 to 4, 3.4 to 4.5, 3.4 to 4.4, 3.4 to 4.3, 3.4 to 4.2, 3.4 to 4.1, 3.4 to 4, 3.5 to 4.5, 3.5 to 4.4, 3.5 to 4.3, 3.5 to 4.2, 3.5 to 4.1, 3.5 to 4, 3.6 to 4.5, 3.6 to 4.4, 3.6 to 4.3, 3.6 to 4.2, 3.6 to 4.1 or 3.6 to 4.

In particular embodiments, the ADC has an average DAR of 3.3 to 4.3, 3.4 to 4.2, 3.5 to 4.1, 3.6 to 4 or 3.7 to 3.9. In a particular embodiment, the ADC has an average DAR of 3.3 to 4.3. In a particular embodiment, the ADC has an average DAR of about 3.8. An average DAR of about 3.8 may encompass an average DAR of e.g. 3.75 to 3.85, 3.76 to 3.84, 3.77 to 3.83, 3.78 to 3.82, 3.79 to 3.81. In some embodiments, the average DAR is 3.80.

As noted above, the cytotoxic agent is generally attached to the antibody via thiol groups of its cysteine residues. In naturally occurring antibodies, many cysteine thiol groups are unavailable for conjugation to other moieties (such as drug molecules) as they exist in the context of disulphide bridges. In some cases, additional reactive thiol groups can be generated by treating the antibody with a reducing agent such as dithiothreitol (DTT) or tris(2-carboxyethyl)phosphine (TCEP), thereby increasing the obtainable DAR.

The ADC may be used in the context of a salt of the ADC described above, e.g. a salt of an inorganic acid such as a hydrochloride, hydrobromide, hydroiodide, nitrate, bicarbonate, carbonate, sulfate or phosphate salt; or a salt of an organic acid salt such as a formate, acetate, propionate, benzoate, maleate, fumarate, succinate, tartrate, citrate, ascorbate, α-ketoglutarate, α-glycerophosphate, alkyl sulfonate or aryl sulfonate salt. Examples of suitable alkyl sulfonate salts include methanesulfonate and ethanesulfonate. Examples of suitable aryl sulfonate salts are benzenesulfonate and p-toluenesulfonate.

The ADC, or salt thereof, may be used in the context of a solvate. The term “solvate” refers to solid or liquid forms of the ADC formed by coordination of the ADC with solvent molecules. A particularly suitable form of the ADC for use herein is a hydrate, which is a solvate with coordinated water molecules.

In some embodiments, the ADC described herein comprises an antibody or antigen-binding fragment thereof that specifically binds claudin 18.2 (CLDN18.2) conjugated to a cytotoxic agent, wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein: V_{H}CDR1 comprises the amino acid sequence set forth in SEQ ID NO: 1; V_{H}CDR2 comprises the amino acid sequence set forth in SEQ ID NO: 2; V_{H}CDR3 comprises the amino acid sequence set forth in SEQ ID NO: 3; V_{L}CDR1 comprises the amino acid sequence set forth in SEQ ID NO: 4; V_{L}CDR2 comprises the amino acid sequence set forth in SEQ ID NO: 5; and V_{L}CDR3 comprises the amino acid sequence set forth in SEQ ID NO: 6.

### Subjects

The subject is a human suffering from biliary tract cancer (BTC) (i.e. a human that has been diagnosed with BTC). The human subject may be referred to as a patient. As used herein, the term “subject” is understood to refer to a “human subject”. In other words, the terms “subject” and “human subject” are used interchangeably.

As used herein, the term “cancer” refers to “biliary tract cancer” or “BTC” unless otherwise indicated or clear from the context. By “biliary tract cancer” is meant any malignancy arising from the biliary tract, i.e. from a bile duct or from the gallbladder (including the cystic duct).

The cancer suffered by the subject expresses CLDN18.2 (by which is meant that at least some of the cells of the cancer express CLDN18.2). In some embodiments, the cancer expresses CLDN18.2 at a particular level, relating to e.g. the proportion of cells which express CLDN18.2 and/or the intensity with which they express the protein. Such levels may be referred to as “thresholds”, since in these embodiments anti-CLDN18.2 therapy is only administered if the patient's cancer expresses CLDN18.2 at or above the specified level. The level of CLDN18.2 expression in the cancer may correlate with the susceptibility of the cancer to anti-CLDN18.2 therapy according to the methods provided herein.

As discussed further below, the level of CLDN18.2 expression in a cancer (or by a cancer) may be determined by analysing a sample of the cancer, e.g. a biopsy. The sample of the cancer may be analysed by any suitable method known in the art capable of measuring the level of expression of a particular protein or gene, e.g. immunohistochemistry (IHC), qPCR or mass spectrometry. Thus, expression of CLDN18.2 may be determined by detection of the CLDN18.2 protein itself, or by detection of mRNA encoding CLDN18.2.

In some embodiments, CLDN18.2 is expressed by at least 20% of cells in the cancer. Thus, the subject treated according to the methods provided herein may have a biliary tract cancer in which CLDN18.2 is expressed by at least 20% of the cells.

In other embodiments, CLDN18.2 is expressed by at least 25% of cells in the cancer. Thus, the subject treated according to the methods provided herein may have a biliary tract cancer in which CLDN18.2 is expressed by at least 25% of the cells.

As noted above, determination of the proportion of cells in a cancer which express CLDN18.2 can be made by analysis of a sample of cells (a tissue sample) from the cancer. Suitable tissue samples include biopsy samples and samples taken from tumours (or parts thereof) which have previously been surgically removed. It can be assumed that the proportion of cells in a cancer which express CLDN18.2 corresponds to the proportion of cells which express CLDN18.2 in a sample taken from that cancer. Thus if at least 20% or 25% of cells in a cancer sample express CLDN18.2, it can be assumed that at least 20% or 25% of cells in the cancer as a whole express CLDN18.2. Thus, while the thresholds set out above refer to the proportion of cells in the cancer expressing CLDN18.2, the thresholds may instead be considered to refer to the proportion of cells in a sample of the cancer expressing CLDN18.2.

Any suitable technique can be used to determine the proportion of cells in a cancer which express CLDN18.2. Most commonly, immunohistochemistry (IHC) is used. Methods of performing IHC are well known in the art and are described in e.g. Kim et al., 2016 and Hofman et al., 2013. The IHC score may be determined using quantitative continuous scoring (QCS), as described in e.g. Kapil et al., 2024.

CLDN18.2 can be detected by IHC using either a monoclonal or polyclonal anti-CLDN18.2 antibody, or an anti-pan-CLDN18 antibody which targets both CLDN18.1 and CLDN18.2. Suitable antibodies for detecting CLDN18.2 are commercially available from various sources, e.g. Abcam and Thermo Fisher Scientific. For instance, the Abcam anti-CLDN18.2 antibodies EPR19202 (catalogue #222512) and EPR19202-244 (catalogue #241330) are suitable for CLDN18.2 detection in IHC. Suitable anti-pan-CLDN18 antibodies include 34H14L15 (Thermo Fisher, catalogue #700178), LS-B16145 (LSBio), NBP-32002 (Novus Biologicals), HPA018446 (Sigma-Aldrich) and 38-8000 (Thermo Fisher). Roche's anti-pan-CLDN18 Ventana antibody (43-14A) is also suitable for CLDN18 detection in IHC. Other suitable antibodies for detecting CLDN18.2 by IHC may be generated by routine methods in the art, e.g. immunization of a small animal such as a mouse or rabbit to generate a polyclonal antibody, or via a hybridoma to obtain a monoclonal antibody. As is well known in the art, in IHC the target protein (e.g. CLDN18.2) is detected using a labelled antibody. The IHC may be performed using a single, labelled primary antibody, or using an unlabelled primary antibody and a labelled secondary antibody. Suitable detectable labels for IHC are known in the art and include fluorescent and chromogenic labels. IHC may be performed manually or may be automated.

Commonly, IHC staining is scored using a semi-quantitative technique, whereby the staining intensity is assigned a score (level) between 0 and 3. According to this scoring system, a score of ‘0’ indicates the staining is negative, i.e. the target protein is not detected; a score of ‘1’ is defined as staining with weak intensity; a score of ‘2’ is defined as staining with moderate intensity; and a score of ‘3’ is defined as staining with strong intensity. This scoring system is well known in the art, as described in e.g. Kim et al., supra; and Rizzardi et al., 2012, and is used in the clinical practice guidelines of the American Society for Clinical Oncology and College of American Pathologist (ACO/CAP) guidelines (see e.g. Wolff et al., 2018), and can thus be routinely applied by a qualified pathologist, e.g. a pathologist certified by the American Board of Pathology, a member of the UK Royal College of Pathologists or a member of any equivalent body elsewhere. Indeed, the scoring system has been commonly used in this or similar form for over 3 decades (see e.g. McCarty et al., 1986).

According to the present methods, the CLDN18.2 expression threshold for treatment with the ADC may include a factor relating to the intensity of IHC staining for CLDN18.2 in the cancer, based on the scoring system described above. Intensity scoring of a cancer sample can readily be performed by a skilled pathologist.

In some embodiments, CLDN18.2 expression is analysed by IHC to determine the proportion of cells in a cancer which express CLDN18.2, but intensity scoring is not performed, or is not factored into the assessment of whether a patient is likely to respond to treatment with the ADC. Thus, in these embodiments, susceptibility to treatment with the ADC is assessed purely based on the proportion of cancer cells which express CLDN18.2.

Thus in these embodiments, at least 20% or 25% of cells in the cancer express CLDN18.2, regardless of IHC staining intensity, i.e. at an IHC staining intensity score of at least 1 (i.e. at an IHC staining intensity score of 1 to 3, that is to say an IHC staining intensity score of 1, 2 or 3). Thus, the subject treated according to the methods provided herein may have a cancer in which CLDN18.2 is expressed by at least 20% or 25% of the cells at an IHC staining intensity of at least 1.

In particular embodiments, the threshold for treatment with the ADC according to the present methods is set at CLDN18.2 expression by at least 25% of cells in the cancer at any IHC staining intensity. That is to say, the subject treated according to the methods provided herein may have a biliary tract cancer in which CLDN18.2 is expressed by at least 25% of the cells at any IHC staining intensity.

According to the therapeutic methods provided herein, whether the cancer expresses CLDN18.2, and if so at what level, may already have been determined, e.g. by IHC as described above. In this case, the therapy is for a subject with a biliary tract cancer which has been determined to express CLDN18.2; or for a subject with a biliary tract cancer the cells of which have been determined to express CLDN18.2.

In particular embodiments, the subject according to the methods provided herein has BTC, wherein at least 20% of cells in the cancer have been determined to express CLDN18.2. In particular embodiments, the subject according to the methods provided herein has BTC, wherein at least 25% of cells in the cancer have been determined to express CLDN18.2. In such embodiments, the CLDN18.2 expression may have been identified by IHC, in which case the at least 20 or 25% of cells in the cancer may have been determined to express CLDN18.2 at a staining intensity level of at least 1.

Alternatively, the therapeutic methods provided herein may include a step of identifying whether a human subject has a biliary tract cancer which is susceptible to treatment with the ADC. Such a step includes: (i) determining whether the cancer expresses CLDN18.2; and (ii) identifying the subject as susceptible to treatment with the ADC if the cancer expresses CLDN18.2.

Suitable tissue samples for assessment of CLDN18.2 expression by the cancer are described above. Assessment of the level of CLDN18.2 expression may be performed by any suitable method, particularly IHC, as described above. Determination of whether the subject is susceptible to treatment with the ADC can be made based on whether the level of CLDN18.2 expression in the tissue sample is above a pre-defined threshold, as described above (e.g. whether at least 20% or 25% of the cells in the cancer express CLDN18.2).

If the subject is determined to be susceptible to treatment with the ADC, the subject is then treated with the ADC as described herein.

Accordingly, in one aspect provided herein is a method of identifying and treating a human subject with biliary tract cancer susceptible to treatment with an ADC described herein, the method comprising:
(a) determining whether the cancer expresses CLDN18.2;
(b) identifying the subject as susceptible to treatment with the ADC if the cancer expresses CLDN18.2; and
(c) when the subject is identified as susceptible to treatment with the ADC, administering an effective amount of the ADC to the subject.

In some embodiments, such a method further comprises an initial step of obtaining the tissue sample from the cancer. As noted above, cancer tissue samples can be obtained e.g. by biopsy or surgery.

In particular embodiments, the biliary tract cancer is cholangiocarcinoma (for example intrahepatic cholangiocarcinoma or extrahepatic cholangiocarcinoma) or gallbladder carcinoma. In some embodiments, the BTC is intrahepatic cholangiocarcinoma. In some embodiments, the BTC is extrahepatic cholangiocarcinoma. The extrahepatic cholangiocarcinoma may be perihilar cholangiocarcinoma or distal cholangiocarcinoma. In some embodiments, the BTC is gallbladder carcinoma, which may arise from cells of the gallbladder or the cystic duct. As noted above, these BTCs express CLDN18.2 with high frequency. As defined herein, ampullary cancer (arising from the ampulla of Vater) is not defined as a biliary tract cancer.

The cancer may be of any stage, but in particular may be an advanced cancer, e.g. a stage III or stage IV cancer. For example, the cancer may be a metastatic cancer or a locally unresectable cancer. In other embodiments, the cancer may be a borderline resectable cancer. In some embodiments, the cancer is unresectable and/or metastatic. Cancer stages referred to herein are those of the TNM staging system, which is well known in the art (see e.g. Rosen et al., 2023).

The subject may be at any stage of treatment. In particular embodiments, the subject is at a second or third stage of treatment. In other words, in such embodiments the subject has received at least one prior line of treatment. In particular embodiments, the at least one prior line of treatment is at least one prior line of systemic treatment. In particular embodiments, the subject has received one or two prior lines of treatment (optionally systemic treatment). In particular embodiments, the ADC used herein is used as a second line treatment (i.e. the subject may have received one prior line of treatment, optionally systemic treatment, for their cancer) or a third line treatment (i.e. the subject may have received two prior lines of treatment, optionally systemic treatment, for their cancer). In particular embodiments, the subject has received a maximum of two prior lines of treatment (optionally systemic treatment) for their cancer. In particular embodiments, the subject has received a maximum of two prior systemic treatments.

By “a line of treatment” as used herein is meant a course of therapy with a drug or a combination of two or more drugs. References to systemic treatment relate to therapy with a drug which is administered via the bloodstream and thus reaches cells throughout the body (as opposed to treatment which is administered locally to the tumour and therefore only reaches cells within the tumour and its immediate vicinity). E.g. a drug administered intravenously is a systemic treatment, whereas a drug administered intratumourally may not be.

In some embodiments, the at least one prior systemic treatment included: a) immunotherapy, optionally wherein the immunotherapy comprised administration of a PD-1 or PD-L1 inhibitor and/or was administered in combination with chemotherapy; and/or b) combination chemotherapy, optionally wherein the combination chemotherapy comprised a fluoropyrimidine and irinotecan or oxaliplatin; and/or c) a targeted therapy, optionally wherein the targeted therapy was an FGFR2 or IDH1 inhibitor.

The one or more prior lines of systemic treatment may have been discontinued for any reason, e.g. patient intolerance, adverse effects or loss of efficacy. In some embodiments, the subject experienced disease progression during or after the at least one prior line of treatment/systemic treatment.

### Treatment Regimens

The subject treated with the ADC provided herein is administered a therapeutically effective dose of the ADC. The term “therapeutically effective” dose is defined as an amount sufficient to achieve or at least partially achieve a desired therapeutic effect. In particular, a therapeutically effective dose may be sufficient to at least temporarily halt disease progression (i.e. such that the cancer patient has stable disease), or alternatively to reduce the speed of disease progression. In some cases, a therapeutically effective dose induces disease regression, i.e. a partial response (PR) or complete response (CR). Exemplary effective doses are described below.

Generally, the subject treated with the ADC is administered at least one dose of the ADC of at least about 1.2 mg/kg. In some embodiments, the subject treated with the ADC is administered at least one dose of the ADC of about 1.2 mg/kg to about 2.2 mg/kg. In particular, the dose may be about 1.2 mg/kg, 1.8 mg/kg or 2.2 mg/kg. In particular embodiments, the dose is about 1.8 mg/kg or 2.2 mg/kg. Generally, the subject treated with the ADC is administered more than one dose of the ADC.

For subjects receiving more than one dose of the ADC, the frequency of the dosing may be determined by the subject's physician, but in general a dose of the ADC may be administered to the subject about every 2 weeks, every 3 weeks, every 4 weeks, every 5 weeks, every 6 weeks, every 7 weeks, every 8 weeks or every 9 weeks. Generally, a dose of the ADC is administered to the subject about every 3 to 9 weeks. In particular embodiments, a dose of the ADC is administered to the subject about every 3 weeks.

By “about” a certain number of weeks, is meant that number of weeks plus or minus 3 days. Thus, a second dose administered about 3 weeks after the first dose may be administered 18 to 24 days after the first dose. In other embodiments, the ADC may be administered to the subject every three weeks, every 3 weeks plus or minus 3 days, plus or minus 2 days, or plus or minus 1 day. While in an ideal scenario the doses might be administered to the subject exactly every 3 weeks, in practice this is not always possible, e.g. if a subject is unwell or unavailable on a given date, and therefore some flexibility in the dosing schedule is advantageous.

In particular embodiments, the ADC is administered about every 3 to 9 weeks, in a dosing schedule whereby the ADC is administered every 3 weeks, unless a delay is necessitated by a treatment-related adverse event, in which case a dose delay of up to 6 weeks is permitted.

When determining a dosing schedule, the date of each dose may be calculated from the date of the prior dose. That is to say, where the ADC is administered about every 3 weeks, the second dose is administered 18 to 24 days after the first dose, the third dose is administered 18 to 24 days after the second dose, and so on. Alternatively, the date of each dose may be calculated from the date of the first dose. In this instance, where the date of the first dose is defined as day 0, and doses are administered about every 3 weeks, the second dose would be administered between days 18 and 24, the third dose would be administered between days 39 and 45, and so on.

In some cases, the lengths between doses in a particular dosing schedule may be altered, if judged to be indicated by the subject's physician. For example, the length of time between doses may be increased to reduce the level of side effects, or reduced for a greater effect, if considered appropriate.

As noted above, the subject treated with the ADC is administered at least one dose of the ADC which may be of about 1.2 mg/kg to about 2.2 mg/kg. In particular embodiments, the at least one dose is of about 1.8 mg/kg or 2.2 mg/kg. Where the subject is administered more than one dose of the ADC, generally the same dosage is given each time. That said, the dosage may be increased or decreased if indicated, as judged by the subject's physician. For example, the dosage may be decreased to reduce the level of side effects, or increased for a greater effect, if considered appropriate.

The dose, or each dose, administered to the subject, may be of about 1.2 to about 2.2 mg/kg, about 1.5 to about 2.2 mg/kg or about 1.8 to about 2.2 mg/kg. In some embodiments, the subject may be administered one or more doses of the ADC of about 1.2 mg/kg, about 1.5 mg/kg, about 1.8 mg/kg or about 2.2 mg/kg. In particular, the subject may be administered one or more doses of the ADC of about 1.8 mg/kg or about 2.2 mg/kg.

In particular embodiments, the subject is administered a dose of the ADC of about 1.8 mg/kg or 2.2 mg/kg, about every 3 weeks.

As noted above, if a patient receiving treatment with the ADC provided herein experiences treatment-related toxicity, the dose may be reduced. If a patient is receiving about 2.2 mg/kg of the ADC every 3 weeks and experiences treatment-related toxicity, the dosage may be reduced to about 1.8 mg/kg every 3 weeks. If a patient is receiving about 1.8 mg/kg of the ADC every 3 weeks (either as a starting dose or reduced from a higher dose) and experiences treatment-related toxicity, the dosage may be reduced to about 1.2 mg/kg every 3 weeks.

By “about” a certain dosage value is meant the specified value±10%. All references to “about” a certain dosage specifically encompass the specified dosage, for example “about 2.2 mg/kg” specifically includes the value of 2.2 mg/kg.

As set out above, the dosages of the ADC used herein are defined as mg/kg. The ‘kg’ here refers to the body mass of the subject to be treated in kilograms. Thus, for example, a dosage of 2.2 mg/kg means 2.2 mg per kg of the body mass of the subject. For example, a subject weighing 70 kg and receiving a dosage of 2.2 mg/kg would receive 154 mg of the ADC.

As noted above, the subject is generally administered more than one dose of the ADC, e.g. at least 2, 3, 4, 5, 6, 7, 8, 9, 10, 12, 14, 16, 18, 20, 22, 24, 26, 28 or 30 doses. For example, in particular embodiments, the subject is administered at least 5, 10, 15, 20, 25 or 30 doses. The number of doses referred to here is the total number of doses administered to the subject through a course of therapy.

The subject may be administered the ADC for a duration of about 1 to 24 months. That is to say, the course of treatment with the ADC may last for about 1 to 24 months, i.e. the final dose of the ADC may be administered to the subject about 1 to 24 months after the first dose. For example, the subject may be administered the ADC for a duration of about 1 to 3, 1 to 4, 1 to 5, 1 to 6, 1 to 7, 1 to 8, 1 to 9, 1 to 10, 1 to 11, 1 to 12, 1 to 15, 1 to 18, 1 to 24, 3 to 6, 3 to 9, 3 to 12, 3 to 15, 3 to 18, 3 to 24, 6 to 9, 6 to 12, 6 to 18, 6 to 24, 12 to 18, or 12 to 24 months. In some cases, the duration of treatment may be longer that 24 months, longer than 30 months or even longer than 36 months. The duration of treatment will vary between patients, with treatment being commenced and halted at the appropriate time for each subject, as determined by their physician. The longer the duration of treatment with the ADC, the more doses will be administered to the subject.

Generally, the course of treatment will last until confirmed disease progression in the subject, unacceptable toxicity to the subject or death of the subject. Disease progression may be determined according to RECIST classification (e.g. RECIST version 1.1, Eisenhauer et al., 2009). Confirmed disease progression may indicate that the ADC is not, or is no longer, effective in treating the subject's biliary tract cancer. Toxicity of the ADC to the subject may be considered unacceptable if the negative impact of the treatment side effects is greater than its anti-cancer benefit. In any event, confirmed disease progression or unacceptable toxicity can be readily determined by the subject's physician.

The length of the course of treatment (e.g. the time until confirmed disease progression, unacceptable toxicity or death) may be dependent on the type of cancer suffered by the subject, the stage of the cancer at the beginning of therapy and/or the line of treatment for which the ADC is used.

Generally, the length of the course of treatment (e.g. the time until confirmed disease progression, unacceptable toxicity or death) may also be dependent on how early in the course of the disease the ADC is administered to the subject. The earlier in treatment that the ADC is administered to the subject, the longer the treatment with the ADC will generally last. That is to say, where the ADC is administered as a second line treatment (such as a second line systemic treatment), the treatment course is likely to last longer than where the ADC is administered as a third line treatment (such as a third line systemic treatment).

Indeed, the length of treatment duration is likely to be impacted by the combination of the line of treatment as which the ADC is used and the type of cancer to be treated.

The ADC described herein may be administered in the context of a monotherapy, by which is meant that the ADC is not administered in combination with any other anti-cancer drug. That is to say, when the ADC is administered as a monotherapy, no other anti-cancer drug is administered to the subject during the course of therapy using the ADC. As would be understood by the skilled person, the ADC monotherapy described herein may be administered concurrently with other drugs for other purposes or to treat other conditions. In some embodiments, the subject may be premedicated with acetaminophen (paracetamol), histamine (H1 and H2)-receptor antagonists and/or steroids, prior to the first dose of the ADC. In some embodiments, the subject may be administered an anti-emetic prior to each dose of the ADC.

Administration of the ADC to the subject may be by any suitable route. Generally, the ADC is administered parenterally, e.g. intravenously, intramuscularly, topically or subcutaneously. Generally, the ADC is administered intravenously.

### Compositions

The ADC for use herein is generally administered in the context of a pharmaceutical composition. A pharmaceutical composition may comprise at least one pharmaceutically acceptable diluent, carrier or excipient, in addition to the ADC. The term “pharmaceutically acceptable” as used herein refers to ingredients that are compatible with other ingredients of the compositions as well as physiologically acceptable to the recipient. The nature of the composition and carriers or excipient materials may be selected in routine manner.

The pharmaceutical composition may be in any form known in the art, but may particularly be a liquid solution of the ADC. Suitable pharmaceutically acceptable diluents, carriers and excipients for inclusion in such liquid solutions are well known in the art. For instance, suitable diluents, carriers and excipients include sucrose, lactose, trehalose, glucose (and other sugars), polyols, liposomes, polyvinyl alcohol, mannitol, gelatin and alcohols.

Liquid pharmaceutical compositions, whether they be solutions, suspensions or other like form, may include one or more of the following: sterile diluents such as water for injection, Ringer's solution, isotonic sodium chloride, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as dextrose. A parenteral preparation can be enclosed in ampoules, disposable syringes or multiple dose vials made of glass or plastic.

Methods of Identifying or Selecting Subjects, Predicting Responses and Assessing Susceptibility to Treatment with the ADC

The level of CLDN18.2 expression in a subject's biliary tract cancer can be assessed in order to identify a subject as susceptible to treatment with the ADC provided herein, to predict whether a subject with cancer is likely to respond to treatment with the ADC provided herein, and/or to identify a subject with cancer who is likely to respond to treatment with the ADC provided herein. Assessment of the level of CLDN18.2 expression in a subject's cancer can be performed as described above. In particular the level of CLDN18.2 expression can be assessed in a tissue sample from the biliary tract cancer. Generally, the level of CLDN18.2 expression in the cancer is assessed by immunohistochemistry (IHC) performed on a tissue sample from the BTC. A subject is considered susceptible to treatment with the ADC if their cancer expresses CLDN18.2. In particular embodiments, the subject is considered susceptible to treatment with the ADC if the level of CLDN18.2 expression in the cancer is at a level as described herein above.

A subject is defined as likely to respond to treatment with the ADC if their BTC is deemed susceptible to such treatment. Similarly, subjects can be selected for treatment with the ADC if their BTC is deemed susceptible to such treatment.

Subjects identified as or predicted to be likely to respond to treatment with the ADC, or with a BTC deemed susceptible to treatment with the ADC, can be selected for treatment with the ADC. Subjects selected for treatment with the ADC can be treated with the ADC, e.g. in a method as described above.

Many modifications and other embodiments of the disclosures set forth herein will come to mind to one skilled in the art to which these disclosures pertain having the benefit of the teachings presented in the foregoing description. Therefore, it is to be understood that the disclosures are not to be limited to the specific embodiments disclosed and that modifications and other embodiments are intended to be included within the scope of the appended claims.

For the avoidance of any doubt, any theoretical explanations provided herein are provided for the purposes of improving the understanding of a reader. The inventors do not wish to be bound by any of these theoretical explanations.

Units, prefixes and symbols may be denoted in their SI accepted form. Unless otherwise indicated, amino acid sequences are written left to right in amino to carboxy orientation, respectively. Amino acids may be referred to herein by either their commonly known three letter symbols or by the one-letter symbols recommended by the IUPAC-IUB Biochemical Nomenclature Commission.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### FIGURE LEGENDS

FIG. 1 shows the effect of the ADC AZD0901 on 16 patient-derived cholangiocarcinoma xenografts implanted into mice. The graph shows the effect of AZD0901 administered at 5 mg/kg (5 mpk) on tumour xenograft size. The data shows the median percentage change in tumour volume for each xenograft group (n=3 mice in each group) relative to baseline (the initial tumour volume at the time of dosing with the ADC). A reduction in tumour volume of at least 30% compared to baseline (indicated by the dotted line) was deemed a response to the treatment. Error bars indicate standard error of the mean (SEM).

FIG. 2 is the same as FIG. 1, but shows the effect of AZD0901 when administered to mice at a dosage of 3.5 mg/kg (3.5 mpk).

FIG. 3 is the same as FIG. 1, but shows the effect of AZD0901 when administered to mice at a dosage of 2.5 mg/kg (2.5 mpk).

FIG. 4 is the same as FIG. 1, but shows the effect of AZD0901 when administered to mice at a dosage of 1.5 mg/kg (1.5 mpk).

### EXAMPLES

### Example 1—Protocol for Phase II Clinical Trial Investigating Treatment of Biliary Tract Cancer with Azd0901

### Objectives, Endpoints and Estimands

| | | |
|---|---|---|
| Type | Objectives | Endpoints/Estimand description |

Primary

| | | |
|---|---|---|
| Safety | To investigate the safety and | Incidence of AEs and SAEs |
| | tolerability of AZD0901 monotherapy | Changes from baseline in laboratory |
| | in participants with advanced or | parameters, vital signs, ECGs, and |
| | metastatic biliary tract tumours | physical examination. |
| | expressing CLDN18.2. | Rate of AEs leading to discontinuation of |
| | | AZD0901 |
| Efficacy | To evaluate the anti-tumour activity of | ORR according to RECIST v1.1 per |
| | AZD0901 monotherapy in | Investigator's assessment |
| | participants with advanced or | ORR is defined as the proportion of |
| | metastatic biliary tract tumours | participants who have a confirmed CR |
| | expressing CLDN18.2 in terms of | or confirmed PR as determined by the |
| | ORR. | Investigator at local site as per |
| | | RECIST v1.1. |
| | | The analysis will include all dosed |
| | | participants with measurable disease at |
| | | baseline and as specified in the |
| | | substudy. |
| | | Data obtained from first dose up until |
| | | progression, or the last evaluable |
| | | assessment in the absence of |
| | | progression, will be included in the |
| | | assessment of ORR, regardless of |
| | | whether the participant withdraws from |
| | | therapy. Participants who go off |
| | | treatment without a response or |
| | | progression, receive a subsequent |
| | | therapy, and then respond, will not be |
| | | included as responders in the ORR. |
| | | The measure of interest is the estimate of |
| | | ORR. |

Secondary

| | | |
|---|---|---|
| Efficacy | To further evaluate the anti-tumour | OS |
| | activity of AZD0901 monotherapy in | OS is defined as time from date of |
| | participants with advanced or | first dose until the date of death due |
| | metastatic biliary tract tumours | to any cause. |
| | expressing CLDN18.2 by | The analysis will include all dosed |
| | assessment of OS, PFS, DoR, DCR, | participants. |
| | DRR and percentage change in | All deaths will be included, |
| | tumour size. | regardless of whether the |
| | | participant withdraws from therapy |
| | | or receives another anti-cancer |
| | | therapy. |
| | | The measures of interest are the |
| | | median OS and estimates of OS at |
| | | 3, 6, 9, and 12 months. |
| | | Radiological response assessed by the |
| | | Investigator evaluated according to |
| | | RECIST v1.1: |
| | | PFS |
| | | PFS is defined as time from date of |
| | | first dose until progression per |
| | | RECIST v1.1 as assessed by the |
| | | Investigator at local site, or death |
| | | due to any cause. |
| | | The analysis will include all |
| | | dosed/randomised participants, |
| | | regardless of whether the |
| | | participant withdraws from therapy, |
| | | receives another anti-cancer |
| | | therapy, or clinically progresses |
| | | prior to RECIST v1.1 progression. |
| | | However, if the participant |
| | | progresses or dies immediately |
| | | after 2 or more consecutive missed |
| | | visits, the participant will be |
| | | censored at the time of the latest |
| | | evaluable assessment prior to the 2 |
| | | missed visits. |
| | | The measures of interest are the |
| | | median PFS, and estimates of PFS |
| | | at 3, 6, 9, and 12 months. |
| | | DoR |
| | | DoR will be defined as the time |
| | | from the date of first documented |
| | | confirmed response until date of |
| | | documented progression per |
| | | RECIST v1.1 as assessed by the |
| | | Investigator at local site or death |
| | | due to any cause. |
| | | The analysis will include all dosed |
| | | participants with measurable |
| | | disease at baseline who have a |
| | | confirmed response, regardless of |
| | | whether the participant withdraws |
| | | from therapy, receives another |
| | | anti-cancer therapy, or clinically |
| | | progresses prior to RECIST v1.1 |
| | | progression, as specified in the |
| | | individual substudies. |
| | | The measure of interest is the |
| | | median of DoR. |
| | | TTR |
| | | TTR is defined as the time from first |
| | | dose until the first documentation of |
| | | a subsequently confirmed objective |
| | | response per RECIST v1.1 as |
| | | assessed by the Investigator at |
| | | local site. |
| | | The analysis will include all dosed |
| | | participants with measurable |
| | | disease at baseline who have a |
| | | confirmed response, regardless of |
| | | whether the participant withdraws |
| | | from therapy, receives another |
| | | anti-cancer therapy, or clinically |
| | | progresses prior to RECIST v1.1 |
| | | progression, as specified in the |
| | | individual substudies. |
| | | The measure of interest is the |
| | | median TTR. |
| | | DCR |
| | | DCR at 12 weeks is defined as the |
| | | percentage of participants who |
| | | have a confirmed CR or PR or who |
| | | have SD per RECIST v1.1 as |
| | | assessed by the Investigator at |
| | | local site and derived from the raw |
| | | tumour data for at least 11 weeks |
| | | after date of first dose. |
| | | Data obtained from first dose up |
| | | until progression, or the last |
| | | evaluable assessment in the |
| | | absence of progression, will be |
| | | included in the assessment of DCR, |
| | | regardless of whether the |
| | | participant withdraws from therapy. |
| | | Participants who receive a |
| | | subsequent therapy prior to week |
| | | 11 will not be considered to have |
| | | disease control in the analysis. |
| | | The analysis will include all dosed |
| | | participants. |
| | | The measure of interest is the |
| | | estimate of DCR at 12 weeks. |
| | | Tumour size |
| | | Percentage change in tumour size |
| | | from baseline. |
| | | DRR-12 |
| | | DRR-12 is defined as the |
| | | percentage of subjects with a |
| | | durable response at 12 weeks. |
| | | DRR-24 |
| | | DRR-24 is defined as the |
| | | percentage of subjects with a |
| | | durable response at 24 weeks. |
| PK | To characterise the PK of AZD0901 | Serum concentration of AZD0901 (total |
| | monotherapy in participants with | ADC), total antibody (conjugated and |
| | advanced or metastatic biliary tract | unconjugated) and total unconjugated |
| | tumours expressing CLDN18.2. | MMAE |
| | | Serum PK parameters of AZD0901, total |
| | | antibody (conjugated and unconjugated) |
| | | and MMAE including but not limited to |
| | | AUC, Cₘₐₓ, tₘₐₓ, clearance and half-life, |
| | | as data allow. |
| ADA | To determine the immunogenicity of | ADA status will be determined along with |
| | AZD0901 monotherapy in | prevalence and incidence of anti-drug |
| | participants with advanced or | antibodies to AZD0901, and titer |
| | metastatic biliary tract tumours | established. |
| | expressing CLDN18.2 | |

Exploratory

| | | |
|---|---|---|
| PD | To evaluate baseline and/or on- | Clinical activity by baseline and/or on- |
| | treatment ctDNA or other blood- | treatment blood-based biomarkers, such |
| | based biomarkers including protein, | as ctDNA dynamics on-treatment, |
| | DNA and/or RNA, and associations | changes in gene expression, immune |
| | with AZD0901 clinical activity. | response signatures, and/or resistance |
| | | mutations. |
| PRO | To describe patient-reported | Descriptive summary of the proportion of |
| | tolerability of AZD0901 monotherapy | participants experiencing treatment- |
| | in participants with advanced or | related symptoms as measured by |
| | metastatic biliary tract tumours | selected items from the EORTC (i.e. |
| | expressing CLDN18.2. | EORTC IL274) and proportion of |
| | | participants reporting different levels of |
| | | overall side-effect bother as measured |
| | | by the PGI-TT. The proportion of |
| | | participants with maintained or improved |
| | | physical function as measured by the |
| | | EORTC physical function scale. The |
| | | analysis will include all |
| | | dosed/randomised participants. The |
| | | measure of interest will be proportion of |
| | | participants reporting different levels of |
| | | treatment-related symptoms, physical |
| | | functioning, and overall tolerability |
| PRO | To assess patient-reported GHS and | Proportion of participants reporting different |
| | QoL of AZD0901 monotherapy or in | levels of GHS/QoL as measured by the |
| | combination with anti-cancer agents | GHS/QoL scale from the EORTC IL274. |
| | in participants with advanced or | The analysis will include all |
| | metastatic biliary tract tumours | dosed/randomised participants. The |
| | expressing CLDN18.2 | measure of interest will be proportion of |
| | | participants reporting different levels of |
| | | GHS/QoL. |
| Optional | To explore how genetic variations | Exploratory endpoints are related to the data |
| Genomics | may affect clinical parameters, risk | generated from the genetic analysis of |
| | and prognosis of diseases and the | part or all of the participant's genetic |
| | response to medications. | information. |
| | To explore how multi-omics | |
| | variations, including genetics, may | |
| | affect clinical parameters, risk and | |
| | prognosis of diseases and the | |
| | response to medications. | |

ADA = anti-drug antibody; ADC = antibody-drug conjugate; AE = adverse event; AUC = area under curve; CLDN = Claudin; Cₘₐₓ = maximum serum concentration; CR = complete response; ctDNA = circulating tumour DNA; DCR = disease control rate; DLT = dose-limiting toxicity; DoR = duration of response; DRR = durable response rate; ECG = electrocardiogram; EORTC IL = European Organisation for Research and Treatment of Cancer - Item library; GHS = global health status; MMAE = monomethyl auristatin E; ORR = objective response rate; OS = overall survival; PD = pharmacodynamics; PD-L1 = programmed death-ligand 1; PFS = progression-free survival; PGI-TT = Patient Global Impression - Treatment Tolerability; PK = pharmacokinetics; PR = partial response; PRO = patient reported outcomes; RECIST v1.1 = Response Evaluation Criteria in Solid Tumours version 1.1; SAE = serious adverse event; SD = stable disease; Tₘₐₓ = time taken for the drug to reach maximum concentration; TTR = time to response; QoL = Quality of Life.

### Overall Design

This clinical trial will investigate the safety, tolerability, and anti-tumour activity of AZD0901 monotherapy in participants with advanced or metastatic BTC expressing CLDN18.2 that have failed previous treatment with one or 2 lines of systemic therapy. Initially, 30 participants will be dosed with IV AZD0901 monotherapy at either 1.8 mg/kg or 2.2 mg/kg Q3W. Part 2 will open to further characterize the efficacy and safety of AZD0901 in participants with BTC at the same dose level as Part 1 if sufficient activity is observed in Part 1. An additional 70 participants may be enroled in Part 2, allowing up to a total of approximately 100 participants in Parts 1 and 2.

The study will also characterise the PK and immunogenicity of AZD0901 monotherapy, and explore potential biological activity by assessing pharmacodynamic and exploratory biomarkers.

### Inclusion Criteria

Participants are eligible to be included in the study only if all of the following criteria apply:

### Informed Consent

1 Capable of giving signed informed consent.

### Age

2 Participant must be ≥18 years or the legal age of consent in the jurisdiction in which the study is taking place at the time of signing the informed consent form (ICF).

### Type of Participant and Disease Characteristics

3 Participants who are CLDN18.2 positive (defined as at least 25% of tumour cells expressing CLDN18.2 at an IHC staining intensity of 1+), as determined prospectively by central IHC testing at pre-screening from archival tumour collected within past 24 months or from a fresh biopsy.
4 Must have at least one measurable lesion according to RECIST v1.1.
   (a) A previously irradiated lesion can be considered a target lesion if the lesion is progressing and well defined.
   (b) For participants who undergo biopsies at screening and/or treatment, it is preferred, though not required, that the biopsied lesion be distinct from any target lesion used in the RECIST v1.1 evaluation.
Histologically confirmed, unresectable advanced, or metastatic adenocarcinoma of biliary tract, including cholangiocarcinoma (intrahepatic or extrahepatic) and gallbladder carcinoma (NOTE: ampullary cancers are pancreatic cancers and so are not eligible).
6 Documented radiographic or clinical disease progression on or after at least one prior regimen and maximum 2 prior lines of systemic treatment for unresectable or metastatic disease.
   (a) Progression within 6 months/183 days of last dose of prior adjuvant or neoadjuvant therapy (including herceptin, immunotherapy) is considered as equivalent to progression on one regimen for advanced or metastatic disease.
   (b) If one of the components of prior combination therapy is discontinued due to AE and the other continued, this is considered to be ‘one prior regimen’.
   (c) If the prior therapy is discontinued due to poor tolerability or AE and the patient is switched to another therapy with no documented progression, this is considered to be ‘one prior regimen’.
   (d) Change in dose or route of administration (e.g. IV or oral fluoropyrimidine) of prior regimen without progression is considered to be ‘one prior regimen’.
   (e) Participants with actionable genomic alterations (e.g. BRAF V600E mutations, FGFR fusions, human epidermal growth factor receptor 2 [HER-2] or IDH mutations) may be eligible as far as participants have received targeted therapy as per local practice.
7 ECOG performance status of 0 to 1 with no deterioration over the previous 2 weeks prior first day of dosing.
8 Predicted life expectancy of ≥12 weeks in the opinion of the Investigator.
9 Participant is willing and able to comply with the study protocol for the duration of the study including undergoing treatment and scheduled visits and examinations.
10 Adequate organ and bone marrow function as shown in table below:

| Parameter | | Value | |
|---|---|---|---|
| Haematological | Haemoglobin ^{a} | ≥9.0 g/dL | (5.59 mmol/L) |
| | Absolute neutrophil count ^{a} | ≥1.5 × 10⁹/L | (1500 per mm³) |
| | Platelet count ^{a} | ≥100 × 10⁹/L | (100000 per mm³) |
| Hepatic | Total bilirubin | ≤1.5× ULN in the absence of Gilbert's syndrome | |
| | | ≤3× ULN if the participant has Gilbert's syndrome | |
| | ALT and AST | ≤3× ULN (for participants with hepatic metastases, | |
| | | ALT, and AST ≤5× ULN) | |
| | Albumin | ≥2.5 | g/dL |
| Renal | Creatinine clearance ^{b} | ≥45 | mL/minute |

| | | |
|---|---|---|
| Coagulation | INR^{c, d} | ≤1.5× ULN |

^{a} Haematological criteria cannot be met with ongoing or recent blood transfusions or EPO treatment (within 7 days prior to the screening assessment and/or scheduled first dose of study treatment) or require growth factor support (within 21 days prior to the scheduled screening assessment and/or first dose of study treatment).
^{b} As determined by Cockcroft - Gault (using actual body weight; Cockcroft and Gault 1976) or 24 - hour urine creatinine clearance.
^{c}Except for participants receiving anticoagulation therapy (INR dose range 2.0 to 3.0 is considered normal).
^{d}If INR is not available, the sites may substitute with the prothrombin time.
ALT = alanine aminotransferase; AST = aspartate aminotransferase; EPO = erythropoietin; INR = International Normalised Ratio; ULN, upper limit of normal.

### Weight

11 Body weight>35 kg.

### Reproduction/Contraception

12 Females of childbearing potential (FOCBP):
   (a) Must have negative pregnancy test at screening and prior to the AZD0901 infusion of each cycle.
   (b) If sexually active with a non-sterilised male partner, must use at least one highly effective method of birth control from screening and must agree to continue using such precautions for 7 months after the last dose of AZD0901.
   (c) Non-sterilised male partners of FOCBP must use a male condom plus spermicide throughout the period specified above for female participant (Note: male condoms are not reliable as a sole contraception method).
13 Female participants must not breastfeed and must not donate, or retrieve for their own use, ova throughout the period specified above for female participants.
14 Non-sterilised male participants who are sexually active with a FOCBP must use a condom with spermicide from screening and must agree to continue using such precautions for 7 months after the last dose of AZD0901 (Note: male condoms are not reliable as a sole contraception method).
   (a) Female partners (of childbearing potential) of a male participant must also use at least one highly effective method of contraception throughout the period specified.
   (b) In addition, male participants must refrain from fathering a child or donating sperm throughout this period.

### Exclusion Criteria

Participants are excluded from the study if any of the following criteria apply:

### Medical Conditions

1 Clinically significant biliary obstruction that has not resolved before enrolment
2 Unstable or active peptic ulcer disease or digestive tract bleeding including but not limited to clinically significant bleeding in the setting of prior CLDN18.2 directed therapy.
3 Participants with clinically significant ascites that require regular drainage.
4 Active or prior documented autoimmune or inflammatory disorders requiring systemic immunosuppressive treatment (including but not limited to inflammatory bowel disease [e.g. colitis, Crohn's disease], diverticulitis, systemic lupus erythematosus, Wegener's syndrome, myasthenia gravis, Grave's disease, rheumatoid arthritis, hypophysitis, uveitis, autoimmune nephritis, or nephropathy, etc.) within the past 3 years prior to the start of treatment. The following are exceptions to this criterion:
   (a) Vitiligo or alopecia.
   (b) Hypothyroidism (eg, following Hashimoto's disease) stable on hormone replacement.
   (c) Psoriasis or eczema not requiring systemic treatment.
5 A history of drug-induced non-infectious ILD/pneumonitis that has required oral or intravenous steroids. Participants with a history of radiation pneumonitis which has clinically and radiologically resolved and not requiring treatment with steroids may be eligible. Investigator must carefully evaluate the benefit-risk assessment on a case-by-case basis.
6 Central nervous system metastases or CNS pathology including: epilepsy, seizures, paresis, aphasia, or stroke within 3 months prior to consent, severe brain injury, dementia, Parkinson's disease, neurodegenerative diseases, cerebellar disease, severe uncontrolled mental illness, psychosis, CNS involvement of autoimmune diseases. The following are exceptions to this criterion:
   (a) Participants with history of seizures are permitted if no active seizures in last 5 years.
   (b) Participants with brain metastases treated, asymptomatic, stable, and not requiring continuous corticosteroids at a dose of >10 mg prednisone/day or equivalent for at least 4 weeks prior to the first dose of AZD0901.
7 Persistent toxicities (CTCAE Grade≥2) caused by previous anti-cancer therapy, excluding alopecia. Participants with irreversible toxicity that is not reasonably expected to be exacerbated by study intervention may be included (e.g. hearing loss) after consultation with study physician.
8 Peripheral neuropathy, sensory, or motor, ≥Grade 2 at screening.
9 History of thromboembolic events within the past 3 months prior to the scheduled first dose of study intervention.
   Participants with venous thromboembolism without history of pulmonary embolism, who either do not require treatment or who have already been on stable treatment with anticoagulants for 3 months or longer prior to start of study intervention may be enrolled and should be closely monitored.
10 History of another primary malignancy except for:
   (a) Malignancy treated with curative intent with no known active disease for at least ≥2 years before the first dose of study intervention and of low potential risk for recurrence.
   (b) Adequately treated non-melanoma skin cancer or lentigo maligna without evidence of disease.
   (c) Adequately treated carcinoma in situ without evidence of disease.
   (d) Localised non-invasive primary disease under surveillance.
11 Uncontrolled active systemic fungal, bacterial, or other infection (defined as exhibiting ongoing signs/symptoms related to the infection and without improvement, despite appropriate antibiotics or other treatment).
12 Active infection exclusions, including tuberculosis and infections with HBV (verified by known positive HBsAg result), HCV, or HIV (verified by positive HIV-1 or HIV-2 antibodies).
   (a) Known uncontrolled hepatitis B and/or chronic or active hepatitis B with HBV DNA≥100 IU/mL.
      (i) Participants with HBsAg positive are eligible if HBV DNA<100 IU/mL and agrees to start or maintain antiviral treatment.
      (ii) Participants with HBsAg negative and HBV viral load ‘detectable’ are eligible if HBV DNA<100 IU/mL and agrees to start or maintain antiviral treatment.
      (iii) Participants with HBsAg negative, anti-HBC positive, and HBV DNA ‘undetectable’ are eligible.
      (iv) Participants with HBsAg negative, anti-HBc negative, and anti-HBs positive are eligible.
      (v) Participants with HBsAg positive or HBV DNA detectable should receive antiviral prophylactic therapy for the duration of anticancer therapy, as well as for at least 12 months after the last dose of anticancer therapy. Participants should have at least 2 weeks of antiviral prophylaxis before starting study drug.
   (b) Known chronic, active, or uncontrolled hepatitis C, defined as anti-HCV IgM/IgG positive and HCV RNA detectable by polymerase chain reaction. Participants with a history of HCV infection are eligible if they have been treated and cured with an undetectable HCV viral load at least 12 weeks post antiviral treatment of HCV.
   (c) Known HIV infection that is not well controlled. All of the following criteria are required to define an HIV infection that is well controlled: undetectable viral RNA load for 6 months, CD4+count of >350, no history of AIDS-defining opportunistic infection within the past 12 months, and stable for at least 6 months on the same anti-HIV medications.
   (d) Active tuberculosis infection (clinical evaluation that may include clinical history, physical examination, and radiographic findings, or tuberculosis testing in line with local practice).
13 Uncontrolled diabetes or diabetic neuropathy within 3 months prior to the first dose of study intervention.
14 As judged by the Investigator, any evidence of clinically significant diseases listed below:
   Corneal disease (e.g. active keratitis or corneal ulcerations)
   Severe or uncontrolled systemic diseases including uncontrolled bleeding disease and gastrointestinal conditions nausea, vomiting, and diarrhoea that could compromise protocol compliance
   Renal transplant, and has a history of bleeding diatheses (e.g. haemophilia or von Willebrand disease) which in the Investigator's opinion makes it undesirable for the participant to participate in the study.
15 Cardiovascular disorder defined as:
   (a) History of arrhythmia (such as multifocal premature ventricular contractions, bigeminy, trigeminy, ventricular tachycardia), which is symptomatic or requires treatment (NCI CTCAE v5.0 Grade 3); symptomatic or uncontrolled atrial fibrillation despite treatment, or asymptomatic sustained ventricular tachycardia. NOTE: abnormalities in serum electrolytes that can increase the risk of arrhythmic events (i.e. sodium, potassium, calcium, magnesium) should be corrected before starting the study intervention.
   (b) Uncontrolled hypertension defined as systolic blood pressure≥160 mmHg and/or diastolic blood pressure≥100 mmHg.
   (c) Acute coronary syndrome/acute myocardial infarction, unstable angina pectoris, coronary intervention procedure with percutaneous coronary intervention, or coronary artery bypass grafting within 6 months.
   (d) History of brain perfusion problems (e.g. carotid stenosis) or stroke, or transient ischaemic attack in the last 6 months prior to screening.
   (e) Symptomatic heart failure (as defined by New York Heart Association class≥II).
   (f) Prior or current cardiomyopathy.
   (g) Severe valvular heart disease.
   (h) Mean resting corrected QT interval (QTcF)>470 milliseconds,
   (i) Left ventricular ejection fraction <50% by echocardiogram or multigated acquisition
   (j) Other cardiovascular and cerebrovascular diseases that, as assessed by the Investigator, are not suitable for participation in this study.

### Prior/Concomitant Therapy

16 Prior exposure to any MMAE-based ADC.
17 Prior exposure to any CLDN18.2 targeted agents except anti-CLDN18.2 monoclonal antibody (e.g. zolbetuximab).
18 Treatment with any of the following agents and interventions:
   Any other anti-cancer agents within the following time periods prior to the first dose of study intervention:
      Cytotoxic treatment: 21 days.
      Non-cytotoxic drugs: 21 days or 5 half-lives (whichever is shorter).
      Biological products including immuno-oncology agents: 28 days.
      Any investigational agents or study interventions from a previous clinical study: 28 days or 5 half-lives (whichever is shorter).
19 Any concurrent anti-cancer treatment. Concurrent use of hormonal therapy for non-cancer-related conditions (e.g. hormone replacement therapy) is allowed.
20 Palliative radiotherapy with a limited field of radiation within 2 weeks or with wide field of radiation or to more than 30% of the bone marrow within 4 weeks before the first dose of study intervention. Participants who have not recovered from radiotherapy-related toxicity to Grade 1 or baseline will not be eligible.
21 Major surgical procedure (excluding placement of vascular access) or significant traumatic injury within 4 weeks of the first dose of study intervention or an anticipated need for major surgery during the study.
22 Treatment with botanical preparations (e.g. herbal supplements or traditional Chinese medicines) intended to treat the disease under study within 2 weeks prior to the first dose of study intervention.
23 Receipt of live attenuated vaccine within 30 days prior to the first dose of study intervention. Note: Participants should not receive live vaccine while receiving study intervention and up to 90 days after the last dose of study intervention. The COVID-19 vaccination should not be given for 72 hours prior to administration of the first dose of study intervention or during the DLT period.
24 Immunosuppressive medications including, but not limited to, methotrexate, azathioprine, and tumour necrosis factor-α blockers are prohibited. The following are exceptions to this criterion:
   (a) Intranasal, inhaled, topical steroids, or local steroid injections (e.g. intraarticular injection)
   (b) Systemic corticosteroids at physiologic doses not to exceed 10 mg/day of prednisone or its equivalent
   (c) Steroids as premedication for hypersensitivity reactions (e.g. CT scan premedication)
   (d) Use of immunosuppressive medications for the management of novel agent-related AEs
   (e) Use of immunosuppressive medications for the management of chemotherapeutics related AEs
   (f) Temporary use (≤14 days) of corticosteroids for concurrent illnesses if clinically indicated and considered to be essential for the management of non-immunotherapy related events experienced by the participant (e.g. allergic reactions not related to novel agent [e.g. food allergies], exacerbation of chronic obstructive pulmonary disease, or short-term control of symptoms related to cancer or radiation [e.g. pain or nausea]) are acceptable.

### Prior/Concurrent Clinical Study Experience

25 Participants with a known hypersensitivity to study intervention or any of the excipients of the product.

### Other Exclusions

26 Involvement in the planning and/or conduct of the study.
27 Judgement by the Investigator that the participant should not participate in the study if the participant is unlikely to comply with study procedures, restrictions, and requirements.
28 Previous enrolment or randomisation in the present study.
29. For females only-currently pregnant (confirmed with positive pregnancy test) or breast-feeding (including when breast-feeding is interrupted) or planning to become pregnant.

### Lifestyle Considerations

Refrain from consumption of grapefruit or grapefruit juice, Seville oranges, grapefruit hybrids, or quinine (e.g. tonic water) from 7 days before the start of study intervention until after the final dose as these may affect AZD0901 metabolism by inhibiting CYP3A4.
During each dosing session, participants will abstain from alcohol for 24 hours before the start of dosing until after collection of the final PK sample.

### Study Interventions and Concomitant Therapy

| | |
|---|---|
| Arm name | AZD0901 |
| Type | Biologic |
| Dose formulation | AZD0901 is supplied as a lyophilised product |
| | for concentrate for solution for infusion. |
| Unit dose strength(s) | 10 mg/mL |
| Dosage level(s) | 1.8 mg/kg or 2.2 mg/kg |
| Route of administration | IV Infusion |
| Regimen | Q3W |

### Administration of Study Interventions

AZD0901 will be administered using an IV bag containing 0.9% sodium chloride and delivered through an IV administration set with a 0.2 or 0.22 μm in-line filter. AZD0901 will be administered Q3W on Day 1 of every 3-week cycle.

### Efficacy Assessments

Tumour assessments will be based on RECIST v1.1. Standard radiographic imaging using RECIST v1.1 is used to assess both response (in participants with measurable disease) and progression. An objective response as per RECIST v1.1 criteria requires confirmation of PR and CR and must occur no fewer than 4 weeks after initial documentation of PR or CR. Disease progression will be defined as per RECIST v1.1.

Assessments for survival will be conducted every 12 weeks following objective PD or treatment discontinuation. Survival information may be obtained via telephone contact with the participant, participant's family, by contact with the participant's current physician, or local death registries. Additional assessments, including subsequent anti-cancer therapy and time to second progression or death, are to be recorded.

### Example 2—In Vivo Anti-Cancer Activity of AZD0901 in Patient-Derived Xenograft Models

### Aim

Evaluate anti-tumour activity of AZD0901 as monotherapy in patient-derived xenograft models of biliary tract cancer.

### Materials and Methods

### Implantation of Patient-Derived Xenografts

For each model, tumours from the same passage were transplanted subcutaneously into CB17 SCID mice (CB-17/lcr-Prkdcscid/lcrlcoCrl). When donor tumours reached 600-1500 mm³, donor animals were euthanized, and tumours were excised and dissected. Necrotic tissue was removed, and viable tumour was sectioned into ~20 mm³ fragments, placed briefly in culture media, and implanted into recipient mice for the experimental study. All mice within a given experiment were implanted on the same day. When cohort mean tumour volumes reached 100-250 mm³, animals were matched by tumour volume and allocated to treatment or control groups. Dosing commenced on Day 0.

### Group Designation and Dose Levels

AZD0901 efficacy was evaluated in 16 cholangiocarcinoma patient-derived xenograft (PDX) models at Champions Oncology (n=5) and Crown Bioscience (n=11). Eighteen female CB17 SCID mice (CB-17/lcr-Prkdcscid/lcrlcoCrl) were enrolled in the treatment phase and randomized by baseline tumour volume to achieve comparable mean volumes across groups. Mice were dosed intravenously (IV) on the day of randomization when cohort mean tumour volumes reached 100-250 mm³. Group 1 received no treatment. Group 2 received a single 5 mg/kg dose of the isotype control ADC NIP228-vc-MMAE. Groups 3, 4, 5 and 6 received single doses of AZD0901 at 1.5, 2.5, 3.5 and 5 mg/kg, respectively.

| | | Dose level | | |
|---|---|---|---|---|
| Group | Treatment | (mg/kg) ^{a} | ROA | Number of animals |
| 1 | Untreated | NA | IV | 3 (F) |
| 2 | NIP228-vcMMAE | 5 mg/kg | IV | 3 (F) |
| 3 | AZD0901 | 1.5 mg/kg | IV | 3 (F) |
| 4 | AZD0901 | 2.5 mg/kg | IV | 3 (F) |
| 5 | AZD0901 | 3.5 mg/kg | IV | 3 (F) |
| 6 | AZD0901 | 5 mg/kg | IV | 3 (F) |

Dose volume: 10 mL/kg;
F = female;
IV = intravenous;
ROA = route of administration

### Determination of Antitumour Response

Animals were weighed and tumour volumes were measured twice weekly from the day of randomization through to the study end. Tumour volume was evaluated by measuring perpendicular tumour diameters with a calliper and tumour volume was calculated using the following formula:

$$Tumour Volume = [ length ( mm ) \times width {( mm )}^{2} ] / 2$$

The antitumor activity of the test agents AZD0901 and NIP228-vc-MMAE was evaluated as described below.

Percent change in tumour volume was calculated for each individual tumour at the day of the greatest tumour regression from starting tumour volume. If the tumour did not regress over the course of the study, the best percent change in tumour volume was calculated at least one week after the study started (study Day 6). For most PDX models, the best response was observed approximately 3 weeks after dosing. For each individual tumour, the tumour volume at the analysis time point (ATV) was compared to the initial tumour volume from the start of dosing (ITV).

The percent change was calculated as:

$$Tumour Growth ( \% ) = [ ( ATV - ITV ) / ITV ] \times 100.$$

If the tumour volume increased between the ITV and the ATV, the Tumour Growth % will be a positive value. Models were considered to be responsive to the test agents if the percent change in tumour volume from baseline was −30% to −100%, inclusive. Reported and graphed Tumour Growth % are the median Tumour Growth % of the mice on study at the time the ATV was recorded.

The growth of tumours in each experimental group was expressed as the mean tumour volume (mm³)+SEM of the number of animals used.

### AZD0901 Efficacy in Cholangiocarcinoma PDX Models

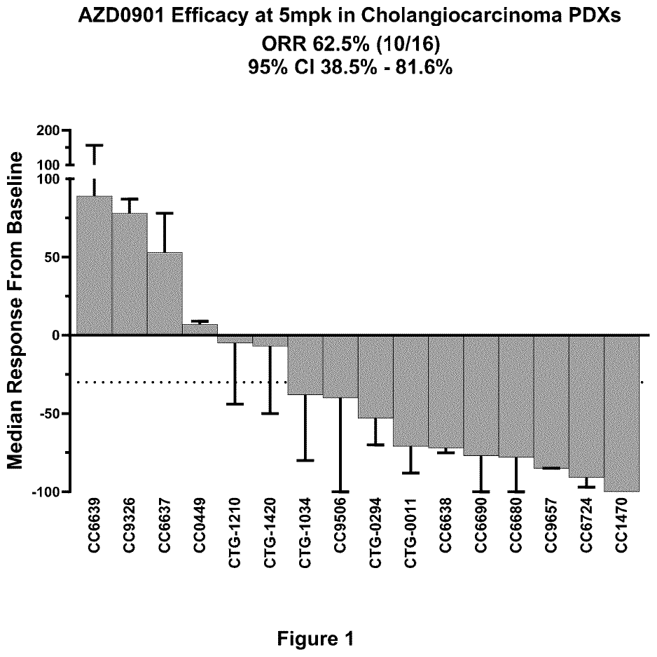

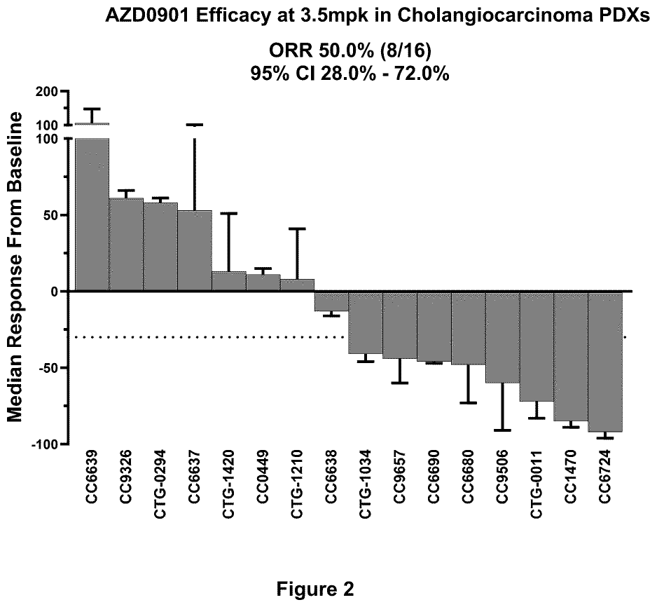

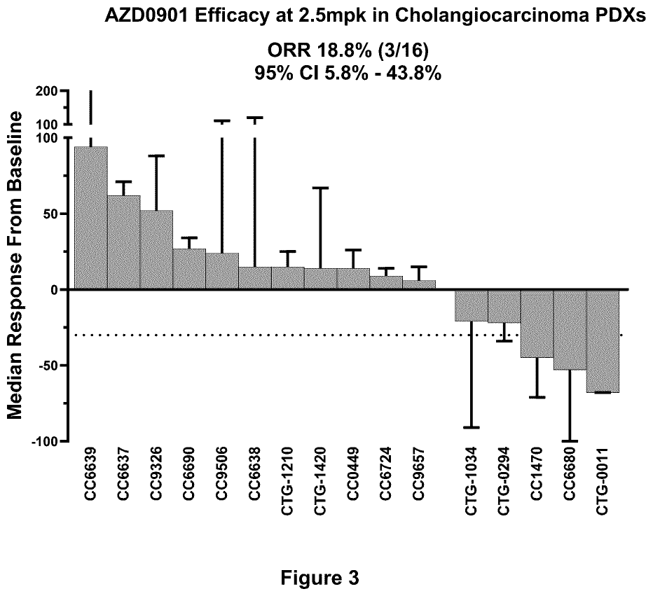

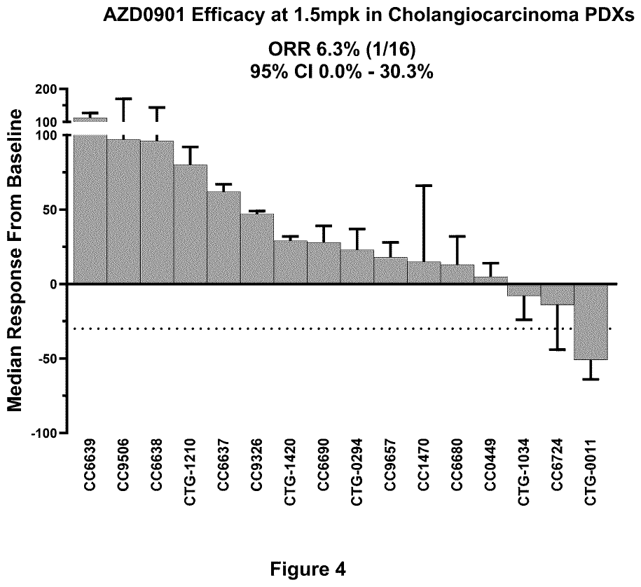

Antitumor activity following single intravenous administrations of 1.5, 2.5, 3.5 and 5 mg/kg AZD0901 monotherapy, compared with untreated control and a 5 mg/kg isotype-control ADC (NIP228-vc-MMAE), is shown in FIGS. 1-4. Tumor growth inhibition—defined as cholangiocarcinoma PDX models exhibiting a reduction in tumour volume from baseline of 30% or greater—was observed with AZD0901 in 1 of 16 models (6.3%) at 1.5 mg/kg, 3 of 16 models (18.8%) at 2.5 mg/kg, 8 of 16 models (50.0%) at 3.5 mg/kg, and 10 of 16 models (62.5%) at 5 mg/kg, indicating dose-dependent antitumour activity. In contrast, a single 5 mg/kg dose of the isotype-control ADC NIP228-vc-MMAE resulted in no models achieving a reduction in tumour volume of 30% or greater from baseline, supporting that the observed antitumor activity of AZD0901 is target-specific.

### SEQUENCE LISTING

All sequences are amino acid sequences.

SEQ ID NO: 1 (CM311 V_{H}CDR1)
GGSISSNYAWN
SEQ ID NO: 2 (CM311 V_{H}CDR2)
YIYYSGNTNYNPSLKS
SEQ ID NO: 3 (CM311 V_{H}CDR3)
SYYGNSFIY
SEQ ID NO: 4 (CM311 V_{L}CDR1)
KSSQSLLNSGNQKNYLT
SEQ ID NO: 5 (CM311 V_{L}CDR2)
WASTRES
SEQ ID NO: 6 (CM311 V_{L}CDR3)
QNAYSFPWT
SEQ ID NO: 7 (CM311 V_{H})
QVQLQESGPGLVKPSETLSLTCTVSGGSISSNYAWNWIRQPPGKGLEWI
GYIYYSGNTNYNPSLKSRVTISRDTSKNQFSLKLSSVTAADTAVYYCAT
SYYGNSFIYWGQGTLVTVSS
SEQ ID NO: 8 (CM311 V_{L})
DIVMTQSPDSLAVSLGERATINCKSSQSLLNSGNQKNYLTWYQQKPGQP
PKLLIYWASTRESGVPDRFSGSGSGTDFTLTISSLQAEDVAVYYCQNAY
SFPWTFGQGTKVEIK
SEQ ID NO: 9 (CM311 heavy chain)
QVQLQESGPGLVKPSETLSLTCTVSGGSISSNYAWNWIRQPPGKGLEWI
GYIYYSGNTNYNPSLKSRVTISRDTSKNQFSLKLSSVTAADTAVYYCAT
SYYGNSFIYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK
SEQ ID NO: 10 (CM311 light chain)
DIVMTQSPDSLAVSLGERATINCKSSQSLLNSGNQKNYLTWYQQKPGQP
PKLLIYWASTRESGVPDRFSGSGSGTDFTLTISSLQAEDVAVYYCQNAY
SFPWTFGQGTKVEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC
SEQ ID NO: 11 (human CLDN18.2)
MAVTACQGLGFVVSLIGIAGIIAATCMDQWSTQDLYNNPVTAVFNYQGL
WRSCVRESSGFTECRGYFTLLGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYV
SEQ ID NO: 12 (human CLDN18.1)
MSTTTCQVVAFLLSILGLAGCIAATGMDMWSTQDLYDNPVTSVFQYEGL
WRSCVRQSSGFTECRPYFTILGLPAMLQAVRALMIVGIVLGAIGLLVSI
FALKCIRIGSMEDSAKANMTLTSGIMFIVSGLCAIAGVSVFANMLVTNF
WMSTANMYTGMGGMVQTVQTRYTFGAALFVGWVAGGLTLIGGVMMCIAC
RGLAPEETNYKAVSYHASGHSVAYKPGGFKASTGFGSNTKNKKIYDGGA
RTEDEVQSYPSKHDYV
SEQ ID NO: 13 (Gly₄Ser linker)
GGGGS

### REFERENCES

A number of publications are cited above in order to more fully describe and disclose the invention and the state of the art to which the invention pertains. Full citations for these references are provided below. The entirety of each of these references is incorporated herein.
Bai et al., J Clin Oncol. 42 (16_suppl): 3028, 2024
Caparica R, Lengele A, Bekolo W, Hendlisz A. FOLFIRI as second-line treatment of metastatic biliary tract cancer patients. Autops Case Rep. 2019; 9 (2): e2019087.
Edgar, R. C., Nucleic Acids Res. 32 (5): 1792-1797, 2004.
Eisenhauer et al., RECIST version 1.1, European Journal of Cancer 45:228-247, 2009.
Fan J, Zhou J, Shi G, Huang X, Gao Q, Liang F et al. Two stage, multi-center trial of cadonilimab and LM-302 for patients with CLDN18.2+biliary tract cancer (BTC) that failed chemotherapy and PD-(L) 1 antibody (ZSAB-Calm). J Clin Oncol. 2024; 42 (16_suppl): e16152.
Hofman & Taylor, Immunohistochemistry Current Protocols in Immunology 103:21.4.1-21.4.26, 2013, John Wiley & Sons, Inc.
Kapil et al., Scientific Reports 14:12129, 2024
Kelley et al., Lancet 401 (10391): 1853-65, 2023
Kim et al., Journal of Pathology and Translational Medicine 50 (6): 411-418, 2016.
Lamarca A, Hubner R A, Ryder W D, Valle J W. Second-line chemotherapy in advanced biliary cancer: a systematic review. Ann Oncol. 2014; 25 (12): 2328-38.
Lamarca A, Palmer D H, Wasan H S, Ross P J, Ma Y T, Arora A et al. Second-line FOLFOX chemotherapy versus active symptom control for advanced biliary tract cancer (ABC-06): a phase 3, open-label, randomised, controlled trial. Lancet Oncol. 2021; 22 (5): 690-701.
McCarty et al., Cancer Research 46 (8-supplement): 4244s-4248s, 1986.
Mizrahi J D, Gunchick V, Mody K, Xiao L, Surapaneni P, Shroff R T et al. Multi-institutional retrospective analysis of FOLFIRI in patients with advanced biliary tract cancers. World J Gastrointest Oncol.
2020; 12 (1): 83-91.
Rice, P. et al., Trends Genet. 16, (6) pp. 276-277, 2000.
Rizzardi et al., Diagnostic Pathology 7:42, 2012.
Rodrigo et al., Antibodies, Vol. 4 (3), p. 259-277, 2015. Antibody fragments which may be used herein include, for example, Fab, F(ab′)2, Fab′ and Fv fragments. Fab fragments are discussed in Nelson, mAbs 2 (1): 77-83, 2010.
Rosen & Sapra, TNM Classification, in: StatPearls, Treasure Island (FL): StatPearls Publishing; 2023 January.
Siegel R L, Miller K D, Jemal A. Cancer statistics, 2020. CA Cancer J Clin. 2020; 70 (1): 7-30.
Sievers F et al., Mol. Syst. Biol. 7:539, 2011.
Takahashi R, Yoshitomi M, Yutani S, Shirahama T, Noguchi M, Yamada A et al. Current status of immunotherapy for the treatment of biliary tract cancer. Hum Vaccin Immunother. 2013; 9 (5): 1069-72.
Williams, D. G. et al., Antibody Engineering Vol. 1, edited by R. Kontermann and S. Dubel, Chapter 21, pp. 319-339, 2010.
Wolff et al., Journal of Clinical Oncology 36 (20): 2105-2122, 2018.
XiaoYi C, Qi C, Gong J, Zhang M, Ma M, Jia K et al (2023). Abstract 6482: CLDN18.2 expression is associated with clinicopathological features and prognosis of Chinese patients with digestive system cancers: a retrospective analysis. Cancer Res. 2023; 83 (7_suppl): 6482.
Xu et al., Journal of Clinical Oncology 41 (4_suppl): 352, 2023
Yoo C, Kim K P, Jeong J H, Kim I, Kang M J, Cheon J et al. Liposomal irinotecan plus fluorouracil and leucovorin versus fluorouracil and leucovorin for metastatic biliary tract cancer after progression on gemcitabine plus cisplatin (NIFTY): a multicentre, open-label, randomised, phase 2b study. Lancet Oncol. 2021; 22 (11): 1560-72.
Yu X, Zhang J, Tazbirkova A, Yang J, Yue J, Sun Y et al. Safety and efficacy of IBI343 (anti-claudin18.2 antibody-drug conjugate) in patients with advanced pancreatic ductal adenocarcinoma or biliary tract cancer: Preliminary results from a phase 1 study. J Clin Oncol. 2024; 42 (16_suppl): 3037

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of treating biliary tract cancer (BTC) in a human subject, comprising administering to the subject a therapeutically effective dose of an antibody-drug conjugate (ADC) comprising an antibody or antigen-binding fragment thereof that specifically binds claudin 18.2 (CLDN18.2) conjugated to a cytotoxic agent,
wherein the antibody or antigen-binding fragment thereof comprises a heavy chain variable region comprising V_{H}CDR1, V_{H}CDR2 and V_{H}CDR3 and a light chain variable region comprising V_{L}CDR1, V_{L}CDR2 and V_{L}CDR3, wherein:
V_{H}CDR1 comprises the amino acid sequence set forth in SEQ ID NO: 1;
V_{H}CDR2 comprises the amino acid sequence set forth in SEQ ID NO: 2;
V_{H}CDR3 comprises the amino acid sequence set forth in SEQ ID NO: 3;
V_{L}CDR1 comprises the amino acid sequence set forth in SEQ ID NO: 4;
V_{L}CDR2 comprises the amino acid sequence set forth in SEQ ID NO: 5; and
V_{L}CDR3 comprises the amino acid sequence set forth in SEQ ID NO: 6;
and wherein the biliary tract cancer expresses CLDN18.2.

2. (canceled)

3. The method of claim 1, wherein the antibody or antigen-binding fragment thereof comprises:
(a) a heavy chain variable domain comprising the amino acid sequence set forth in SEQ ID NO: 7, or a variant thereof having at least 80% identity to SEQ ID NO: 7; and
(b) a light chain variable domain comprising the amino acid sequence set forth in SEQ ID NO: 8, or a variant thereof having at least 80% identity to SEQ ID NO: 8.

4. The method of claim 3, wherein the antibody or antigen-binding fragment thereof comprises:
(a) a heavy chain variable domain comprising the amino acid sequence set forth in SEQ ID NO: 7; and
(b) a light chain variable domain comprising the amino acid sequence set forth in SEQ ID NO: 8.

5. (canceled)

6. The method of claim 1, wherein the antibody comprises:
(a) a heavy chain comprising the amino acid sequence set forth in SEQ ID NO: 9, or a variant thereof having at least 80% identity to SEQ ID NO: 9; and
(b) a light chain comprising the amino acid sequence set forth in SEQ ID NO: 10, or a variant thereof having at least 80% identity to SEQ ID NO: 10.

7. The method of claim 6, wherein the antibody comprises:
(a) a heavy chain comprising the amino acid sequence set forth in SEQ ID NO: 9; and
(b) a light chain comprising the amino acid sequence set forth in SEQ ID NO: 10.

8. The method of claim 1, wherein the cytotoxic agent is monomethyl auristatin E (MMAE).

9. The method of claim 1, wherein the cytotoxic agent is joined to the antibody or antigen-binding fragment thereof via a 6-maleimidocaproyl-valine-citrulline-p-aminobenzyloxycarbonyl (MC-vc-PAB) linker.

10.-11. (canceled)

12. The method of claim 1, wherein the ADC has an average drug-to-antibody ratio (DAR) of 3.3 to 4.3.

13. The method of claim 12, wherein the ADC has an average DAR of 3.8.

14. The method of claim 1, wherein CLDN18.2 is expressed by at least 20% of cells in the biliary tract cancer.

15. The method of claim 14, wherein CLDN18.2 is expressed by at least 25% of cells in the biliary tract cancer.

16. The method of claim 1, wherein the biliary tract cancer, or the cells in the biliary tract cancer, has been determined to express CLDN18.2.

17. The method of claim 16, wherein CLDN18.2 expression has been determined by immunohistochemistry (IHC) and CLDN18.2 is expressed at a staining intensity level of at least 1 by at least 25% of cells in the biliary tract cancer.

18. (canceled)

19. The method of claim 1, wherein the biliary tract cancer is:
(i) intrahepatic cholangiocarcinoma, extrahepatic cholangiocarcinoma or gallbladder carcinoma; and/or
(ii) unresectable and/or metastatic.

20. (canceled)

21. The method of claim 1, wherein the subject has received at least one prior line of systemic treatment, and/or
wherein the subject has received a maximum of two prior lines of systemic treatment.

22-23. (canceled)

24. The method of claim 21, wherein the at least one prior line of systemic treatment included:
(a) immunotherapy, optionally wherein the immunotherapy comprised administration of a PD-1 or PD-L1 inhibitor and/or was administered in combination with chemotherapy; and/or
(b) combination chemotherapy, optionally wherein the combination chemotherapy comprised a fluoropyrimidine and irinotecan or oxaliplatin; and/or
(c) a targeted therapy, optionally wherein the targeted therapy was an FGFR2 or IDH1 inhibitor.

25. The method of claim 21, wherein the subject experienced disease progression during or after the at least one line of systemic treatment.

26. The method of claim 1, wherein the subject is administered at least one dose of the ADC of at least about 1.2 mg/kg.

27. The method of claim 26, wherein the subject is administered at least one dose of the ADC of about 1.2 mg/kg to about 2.2 mg/kg.

28. The method of claim 27, wherein the subject is administered at least one dose of the ADC of about 1.8 mg/kg or 2.2 mg/kg.

29. The method of claim 1, wherein the subject is administered a dose of the ADC about every 3 weeks; and/or
wherein the subject is administered a total of at least 2 doses, at least 3 doses, at least 4 doses, at least 5 doses, or at least 6 doses of the ADC, or is administered the ADC until disease progression, unacceptable toxicity or death.

30-32. (canceled)

33. The method of claim 1, wherein the ADC is administered as a monotherapy.

34-51. (canceled)

52. A method of selecting a human subject with biliary tract cancer for treatment with an ADC as defined in claim 1, the method comprising assessing the level of CLDN18.2 expression in a tissue sample from the biliary tract cancer,
wherein the subject is selected for treatment with the ADC when the cancer expresses CLDN18.2.

53. A method of predicting whether a human subject with biliary tract cancer is likely to respond to treatment with an ADC as defined in claim 1, the method comprising assessing the level of CLDN18.2 expression in a tissue sample from the biliary tract cancer,
wherein the subject is considered likely to respond to treatment with the ADC when the cancer expresses CLDN18.2.

54. A method for identifying a human subject with biliary tract cancer who is likely to respond to treatment with an ADC as defined in claim 1, the method comprising obtaining a tissue sample of the biliary tract cancer from the subject and assessing the level of CLDN18.2 expression in the sample,
wherein the subject is considered likely to respond to treatment with the ADC when the cancer expresses CLDN18.2.

55. A method for assessing the susceptibility of a biliary tract cancer in a human subject to treatment with an ADC as defined in claim 1, comprising assessing the level of CLDN18.2 expression in a tissue sample from the biliary tract cancer,
wherein the biliary tract cancer is considered susceptible to treatment with the ADC when the cancer expresses CLDN18.2.

56.-60. (canceled)
